(19) United States
(12) Patent Application Publication
(10) Pub. No.: US 2026/0106041 A1
(43) Pub. Date: Apr. 16, 2026

(54) METHODS AND SYSTEMS FOR PREDICTING TREATMENT RESPONSE TO MONO-IMMUNOTHERAPY AND CHEMO-IMMUNOTHERAPY

(71) Applicant: Foundation Medicine, Inc., Boston, MA (US)

(72) Inventors: Ericka EBOT, Newton, MA (US); Nataly Moran CABILI, Somerville, MA (US); Lee Alan ALBACKER, Arlington, MA (US)

(73) Assignee: Foundation Medicine, Inc., Boston, MA (US)

(21) Appl. No.: 19/360,287

(22) Filed: Oct. 16, 2025

Related U.S. Application Data
(63) Continuation of application No. PCT/US2024/025206, filed on Apr. 18, 2024.
(60) Provisional application No. 63/460,816, filed on Apr. 20, 2023.

(51) Int. Cl. G16H 50/30; G16B 20/10; G16B 20/20

(52) U.S. Cl. CPC G16H 50/30; G16B 20/10; G16B 20/20

(57) ABSTRACT

Methods for predicting a survival metric to aid health care providers in determine a first line treatment are described. The methods may comprise, for example, receiving sequence read data associated with a sample from the individual, selecting a plurality of sequence reads from the sequence read data, determining one or more characterization features based on the plurality of sequence reads, receiving one or more digital pathology features, inputting, using the one or more processors, the one or more characterization features and the one or more digital pathology features into one or more statistical models, and predicting a survival metric indicative of a likelihood of survival of the individual, based on outputs of the one or more statistical models.

### CROSS-REFERENCE TO RELATED APPLICATION

This application is a continuation application of International Application No. PCT/US2024/025206, filed internationally on Apr. 18, 2024, which claims the benefit of U.S. Provisional Application No. 63/460,816, filed Apr. 20, 2023, the disclosures of which are herein incorporated by reference in their entirety.

### FIELD OF THE INVENTION

The present disclosure relates generally to methods and systems for analyzing genomic profiling data and digital pathology features, and more specifically to methods and systems for predicting a response of an individual to immunotherapy treatment based on the genomic profiling data and digital pathology features.

### BACKGROUND

When an individual presents with cancer, health care providers often turn to mono-immunotherapy or chemo-immunotherapy as a first line treatment. Selecting the optimal first line treatment impacts the individual's overall survival and quality of life. Determining whether to administer mono-immunotherapy or chemo-immunotherapy, however, is not a clear-cut decision and health care providers consider many factors when determining the first line treatment. The sheer number of factors to consider along with the complex interrelated nature of many of these factors complicate the decision-making process for a health care provider as it is often not clear how these various factors will impact a patient's overall survival. Accordingly, there is a need to provide a biomarker that can aid healthcare providers in selecting a first line treatment (e.g., mono-immunotherapy or chemo-immunotherapy and/or combination therapy) for individuals.

### BRIEF SUMMARY OF THE INVENTION

Disclosed herein are methods and systems for determining a survival metric indicative of survival of a patient when treated with either mono-immunotherapy or chemo-immunotherapy.

Embodiments of the present disclosure integrate information about the tissue micro-environment (e.g., corresponding to digital pathology images and/or associated quantitative pathology features (QPFs)) with genomic information (e.g., determined using next-generation sequencing (NGS) and clinical information. In some embodiments the survival metric may correspond to a biomarker that may aid healthcare providers in determining first line treatment decisions of whether to treat a patient with mono-immunotherapy or chemo-immunotherapy. Embodiments of the present disclosure may be applicable to individuals with advanced non-squamous non-small cell lung cancer (NSCLC) patients without actionable alterations in EGFR and ALK. A skilled artisan will understand that the systems and methods described herein may be applied to other types of cancer without departing from the scope of this disclosure.

Embodiments of the present disclosure may also provide systems and methods for providing bias correction regarding determining a patient's estimated overall survival. For instance, there is often a bias based on whether the sample is a biopsy or resection. For example, patients associated with needle core biopsy samples versus tumor resection samples tend to have worst overall survival. The sample type appears to be associated with the overall health of the patient and the extent of their metastatic disease at the time of surgery/sample biopsy. Embodiments of the present disclosure further provide bias correction based on the sample type.

Embodiments of the specification provide methods for predicting a survival of an individual with a disease. In one or more embodiments, the method comprises: providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, at one or more processors, sequence read data for the plurality of sequence reads associated with the sample from the subject; selecting, using the one or more processors, a plurality of sequence reads from the sequence read data; determining, using the one or more processors, one or more characterization features based on the plurality of sequence reads; receiving, using the one or more processors, one or more digital pathology features; inputting, using the one or more processors, the one or more characterization features and the one or more digital pathology features into one or more statistical models; and predicting, using the one or more processors, a survival metric indicative of a likelihood of survival of the individual, based on outputs of the one or more statistical models.

In one or more embodiments, the one or more characterization features comprise a presence of a genomic alteration, an absence of a genomic alteration, a tumor mutational burden (TMB), a PD-L1 status, a chromosome arm level, cytoband level copy number alterations, mutational signatures, copy number signatures, or a combination thereof.

In one or more embodiments, the method further comprises: receiving, using the one or more processors, one or more clinical features; and inputting, using the one or more processors, the one or more clinical features into the one or more statistical models.

In one or more embodiments, the one or more clinical features comprises a sample type and the method further comprises pre-processing the one or more clinical features to account for bias associated with the sample type. In one or more embodiments, the sample type comprises a tissue resection type, a core needle biopsy type, or an unknown type. In one or more embodiments, the one or more characterization features and the one or more digital pathology features are selected based on the disease.

In one or more embodiments, an output of the statistical model comprises a survival score, wherein the survival score is indicative of the survival of the individual. In one or more embodiments, the survival score is indicative of the survival of the individual when treated with a treatment. In one or more embodiments, predicting the survival metric of the individual comprises: comparing the survival score to one or more predefined thresholds, in accordance with a determination that the survival score is greater than or equal to a first predetermined threshold, determining, using the one or more processors, that the survival metric is indicative of a poor likelihood of survival; and in accordance with a determination that the survival score is less than or equal to the first predetermined threshold, determining, using the one or more processors, that the survival metric is indicative of a strong likelihood of survival.

In one or more embodiments, the subject is suspected of having or is determined to have cancer. In one or more embodiments, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In one or more embodiments, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In one or more embodiments, the method further comprises treating the subject with an anti-cancer therapy. In one or more embodiments, the anti-cancer therapy comprises a targeted anti-cancer therapy. In one or more embodiments, the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane 1131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In one or more embodiments, the method further comprises obtaining the sample from the subject. In one or more embodiments, the sample comprises a tissue biopsy sample. In one or more embodiments, the plurality of nucleic acid molecules comprises a mixture of tumor nucleic acid molecules and non-tumor nucleic acid molecules. In one or more embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In one or more embodiments, the one or more adapters comprise amplification primers, flow cell adaptor sequences, substrate adapter sequences, or sample index sequences. In one or more embodiments, the captured nucleic acid molecules are captured from the amplified nucleic acid molecules by hybridization to one or more bait molecules. In one or more embodiments, the one or more bait molecules comprise one or more nucleic acid molecules, each comprising a region that is complementary to a region of a captured nucleic acid molecule.

In one or more embodiments, amplifying nucleic acid molecules comprises performing a polymerase chain reaction (PCR) amplification technique, a non-PCR amplification technique, or an isothermal amplification technique.

In one or more embodiments, the sequencing comprises use of a massively parallel sequencing (MPS) technique, whole genome sequencing (WGS), whole exome sequencing, targeted sequencing, direct sequencing, or Sanger sequencing technique. In one or more embodiments, the sequencing comprises massively parallel sequencing, and the massively parallel sequencing technique comprises next generation sequencing (NGS). In one or more embodiments, the sequencer comprises a next generation sequencer. In one or more embodiments, one or more of the plurality of sequencing reads overlap one or more gene loci within one or more subgenomic intervals in the sample.

In one or more embodiments, the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

In one or more embodiments, the one or more gene loci comprise ABL1, ACVR1B, AKT1, AKT2, AKT3, ALK, ALOX12B, AMERI, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKB1A, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCDILG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKC1, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.

In one or more embodiments, the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3K8, PIGF, PTCH, RAF, RANKL, RET, ROS1, SLAMF7, VEGF, VEGFA, VEGFB, or any combination thereof.

In one or more embodiments, the method further comprises generating, by the one or more processors, a report indicating the survival metric. In one or more embodiments, the method further comprises transmitting the report to a healthcare provider. In one or more embodiments, the report is transmitted via a computer network or a peer-to-peer connection.

Embodiments of the present disclosure further comprise methods for predicting a survival of an individual with a disease. In one or more embodiments, the method comprises: receiving, using one or more processors, sequence read data associated with a sample from the individual; selecting, using the one or more processors, a plurality of sequence reads from the sequence read data; determining, using the one or more processors, one or more characterization features based on the plurality of sequence reads; receiving, using the one or more processors, one or more digital pathology features; inputting, using the one or more processors, the one or more characterization features and the one or more digital pathology features into one or more statistical models; and predicting, using the one or more processors, a survival metric indicative of a likelihood of survival of the individual, based on outputs of the one or more statistical models.

In one or more embodiments, the one or more characterization features comprise a presence of a genomic alteration, an absence of a genomic alteration, a tumor mutational burden (TMB), a PD-L1 status, chromosome arm level, cytoband level copy number alterations, mutational signatures, copy number signatures, or a combination thereof. In one or more embodiments, the PD-L1 status comprises a negative status, a positive-low status, or a positive-high status. In one or more embodiments, the genomic alteration comprises a single nucleotide variant (SNV), insertion and/or deletion (indel), rearrangement, or copy number variant (CNV).

In one or more embodiments, one or more digital pathology features comprise a total cancer epithelium value, a regional cancer epithelium value, an area of a largest region of cancer epithelium, a perimeter of the largest region of cancer epithelium, a filled area of the largest region of cancer epithelium, a number of clusters of cells in the cancer epithelium, or a combination thereof.

In one or more embodiments, the method further comprises: receiving, using the one or more processors, one or more clinical features; and inputting, using the one or more processors, the one or more clinical features into the one or more statistical models. In one or more embodiments, the one or more clinical features comprises a sex, a smoking status, a tissue type, a sample type, a practice type, a self-care functional performance status (ECOG performance status), an age, a stage at diagnosis, a tumor type, or a combination thereof.

In one or more embodiments, the sample type comprises a tissue resection type, a core needle biopsy type, or an unknown type. In one or more embodiments, the one or more characterization features and the one or more digital pathology features are selected based on the disease. In one or more embodiments, the one or more digital pathology features comprise quantitative pathology features (QPFs). In one or more embodiments, the one or more digital pathology features are obtained from one or more whole slide images (WSIs). In one or more embodiments, the one or more clinical features comprises the sample type and the method further comprises pre-processing the one or more clinical features to account for bias associated with the sample type.

In one or more embodiments, the method further comprises reducing a number of the one or more digital pathology features by associating the one more digital pathology features into one or more groups based on highly correlated features of the one or more digital pathology features.

In one or more embodiments, an output of the statistical model comprises a survival score, wherein the survival score is indicative of the survival of the individual. In one or more embodiments, the survival score is indicative of the survival of the individual when treated with a treatment. In one or more embodiments, predicting, using the one or more processors, the survival metric of the individual comprises comparing the survival score to one or more predefined thresholds, in accordance with a determination that the survival score is greater than or equal to a first predetermined threshold, determining, using the one or more processors, that the survival metric is indicative of a poor likelihood of survival; and in accordance with a determination that the survival score is less than or equal to the first predetermined threshold, determining, using the one or more processors, that the survival metric is indicative of a strong likelihood of survival. In one or more embodiments, the strong likelihood of survival is associated with a likelihood of survival of greater than 50% for a predetermined period of time. In one or more embodiments, the poor likelihood of survival is associated with a likelihood of survival of less than 50% for the predetermined period of time.

In one or more embodiments, the method further comprises determining the one or more predetermined thresholds using an F1-score, an F2-score, Mathew's correlation coefficient, Newton's index, a time-dependent ROC curve, a hazard ratio and p-value, a likelihood ratio test, or a combination thereof.

In one or more embodiments, the survival metric is associated with a treatment comprising an immunotherapy. In one or more embodiments, the immunotherapy is a monotherapy. In one or more embodiments, the treatment comprises an immunotherapy in combination with a chemotherapy. In one or more embodiments, the chemotherapy comprises one or more of a platinum agent and an antifolate agent. In one or more embodiments, the platinum agent is cisplatin or carboplatin. In one or more embodiments, the antifolate is pemetrexed. In one or more embodiments, the chemotherapy comprises cisplatin or carboplatin in combination with pemetrexed. In one or more embodiments, the treatment is a first-line treatment. In one or more embodiments, the immunotherapy comprises an immune checkpoint inhibitor (ICI). In one or more embodiments, the ICI comprises an inhibitor of PD-1 or PD-L1.

In one or more embodiments, the one or more statistical models comprises a first statistical model associated with immunotherapy as a monotherapy and a second statistical model associated with immunotherapy in combination with a chemotherapy. In one or more embodiments, an output of the first statistical model corresponds to a survival score indicative of an overall survival or progression free survival of the individual when treated with the immunotherapy as monotherapy and an output of the second statistical model corresponds to a survival score indicative of an overall survival or progression free survival of the individual when treated with the immunotherapy in combination with chemotherapy.

In one or more embodiments, the individual has cancer. In one or more embodiments, the individual has non-small cell lung cancer (NSCLC). In one or more embodiments, the individual has advanced, non-squamous NSCLC. In one or more embodiments, the NSCLC does not have an alteration in an EGFR or ALK gene.

In one or more embodiments, the method further comprises training the statistical model, wherein training the statistical model comprises: receiving, using the one or more processors, training data based on a plurality of training samples; and training, using the one or more processors, the statistical model based on the training data to obtain a trained statistical model.

In one or more embodiments, training the statistical model comprises: inputting, using the one or more processors, the training data into the statistical model; determining, using the one or more processors, a score based on the training data; and updating, using the one or more processors, one or more weights associated with the statistical model based on the score. In one or more embodiments, the one or more weights are associated with a specific disease. In one or more embodiments, the method further comprises filtering, using the one or more processors, the training data to select the one or more characterization features and the one or more digital pathology features for the trained statistical model based on the weights. In one or more embodiments, the training data comprises one or more training characterization features, one or more training digital pathology features, one or more training clinical features, one or more clinical outcomes, or a combination thereof.

In one or more embodiments, the statistical model is a machine learning model. In one or more embodiments, the statistical model is part of a machine learning process. In one or more embodiments, the statistical model includes an artificial intelligence learning model. In one or more embodiments, the statistical model comprises a random forest model. In one or more embodiments, the statistical model comprises a lasso regression model. In one or more embodiments, the statistical model comprises at least one of an extreme gradient boosting model, a logistic regression model, a lasso regression model, an elastic net model, a ridge regression model, a random forest model, a support vector machine model, a k-nearest neighbor model, a Bayesian model, a naïve-based model, a Gaussian naïve-based model, a clustering model, a limited expectation maximization model, a gradient boosting ensemble model, an adaboost model, a bagging model, a neural network model, a backpropagation model, a stochastic gradient descent model, a linear regression model, a non-linear regression model, a multivariate regression model, a robust machine learning model, and a proportional hazards model.

In one or more embodiments, the sequence read data for the individual is based on a targeted exome sequencing panel. In one or more embodiments, the sequence read data for the individual is based on a whole exome sequencing panel or a whole genome sequencing panel. In one or more embodiments, the sequence read data for the individual is derived from a single biopsy sample or derived from multiple biopsy samples. In one or more embodiments, the one or more characterization features comprises: a BCOR alteration, a BRAF alteration, a FAM123B alteration, a GATA3 alteration, a HGF alteration, an IRF2 alteration, a NF2 alteration, a NPM1 alteration, a PIK3CA alteration, a RAD54L alteration, a SDHC alteration, a SMAD4 alteration, and a tumor mutational burden, and wherein the one or more digital pathology features comprises a granulocyte cell count over an immune cell count, a cluster dispersion standard deviation of lymphocytes in a cancer epithelium, a cluster size standard deviation of cancer cells in the cancer epithelium, a solidity of a largest region of a cancer stroma, and a total Euler number of the cancer epithelium.

In one or more embodiments, the method further comprises assigning, using the one or more processors, a therapy for the individual based on the predicted survival metric. In one or more embodiments, the method further comprises administering, using the one or more processors, a treatment to the individual based on the predicted survival metric. In one or more embodiments, the method further comprises associating, using the one or more processors, the individual with a clinical trial based on the predicted survival metric. In one or more embodiments, the method further comprises monitoring, using the one or more processors, a prognosis of the individual based on the predicted survival metric. In one or more embodiments, the method further comprises predicting, using the one or more processors, one or more clinical outcomes based on the predicted survival metric.

Embodiments of the present disclosure further comprise methods for predicting a response to treatment or a survival of an individual with a disease. In one or more embodiments, the method comprises: receiving, using one or more processors, sequence read data associated with a sample from the individual; selecting, using the one or more processors, a plurality of sequence reads from the sequence read data; determining, using the one or more processors, one or more characterization features based on the plurality of sequence reads; receiving, using the one or more processors, one or more digital pathology features; inputting, using the one or more processors, the one or more characterization features and the one or more digital pathology features into one or more statistical models; and predicting, using the one or more processors, a survival metric indicative of a likelihood of survival of the individual or an estimated response to treatment, based on outputs of the one or more statistical models.

Embodiments of the present disclosure further comprise methods for predicting a response to treatment of an individual with a disease. In one or more embodiments, the method comprises: receiving, using one or more processors, sequence read data associated with a sample from the individual; selecting, using the one or more processors, a plurality of sequence reads from the sequence read data; determining, using the one or more processors, one or more characterization features based on the plurality of sequence reads; receiving, using the one or more processors, one or more digital pathology features; inputting, using the one or more processors, the one or more characterization features and the one or more digital pathology features into one or more statistical models; and predicting, using the one or more processors, a survival metric indicative of a estimated response of the individual to the treatment, based on outputs of the one or more statistical models.

Embodiments of the present disclosure further comprise methods for treating a subject having a cancer. In one or more embodiments, the method comprises: receiving, using one or more processors, sequence read data associated with a sample from the individual; selecting, using the one or more processors, a plurality of sequence reads from the sequence read data; determining, using the one or more processors, one or more characterization features based on the plurality of sequence reads; receiving, using the one or more processors, one or more digital pathology features; inputting, using the one or more processors, the one or more characterization features and the one or more digital pathology features into one or more statistical models; predicting, using the one or more processors, a survival metric indicative of a likelihood of survival of the individual, based on outputs of the one or more statistical models; treating the subject with a mono-immunotherapy if the survival metric is above a threshold; and treating the subject with the mono-immunotherapy and a chemo-immunotherapy if the survival metric is below the threshold.

Embodiments of the present disclosure further comprise methods for selecting a treatment for a subject having a cancer. In one or more embodiments, the method comprises: receiving, using one or more processors, sequence read data associated with a sample from the individual; selecting, using the one or more processors, a plurality of sequence reads from the sequence read data; determining, using the one or more processors, one or more characterization features based on the plurality of sequence reads; receiving, using the one or more processors, one or more digital pathology features; inputting, using the one or more processors, the one or more characterization features and the one or more digital pathology features into one or more statistical models; predicting, using the one or more processors, a survival metric indicative of a likelihood of survival of the individual, based on outputs of the one or more statistical models; selecting a mono-immunotherapy as the treatment if the survival metric is above a threshold; and selecting the mono-immunotherapy and a chemo-immunotherapy as the treatment if the survival metric is below the threshold.

Embodiments of the present disclosure further comprise methods for identifying one or more treatment options for a subject having a cancer. In one or more embodiments, the method comprises: receiving, using one or more processors, sequence read data associated with a sample from the individual; selecting, using the one or more processors, a plurality of sequence reads from the sequence read data; determining, using the one or more processors, one or more characterization features based on the plurality of sequence reads; receiving, using the one or more processors, one or more digital pathology features; inputting, using the one or more processors, the one or more characterization features and the one or more digital pathology features into one or more statistical models; and predicting, using the one or more processors, a survival metric indicative of a likelihood of survival of the individual, based on outputs of the one or more statistical models; identifying a mono-immunotherapy as the treatment if the survival metric is above a threshold; and identifying the mono-immunotherapy and a chemo-immunotherapy as the treatment if the survival metric is below the threshold.

Embodiments of the present disclosure further comprise methods for treating a subject having a cancer. In one or more embodiments, the method comprises: receiving, using the one or more processors, one or more digital pathology features; inputting, using the one or more processors, the one or more digital pathology features into one or more statistical models; predicting, using the one or more processors, a survival metric indicative of a likelihood of survival of the individual, based on outputs of the one or more statistical models; treating the subject with a mono-immunotherapy if the survival metric is above a threshold; and treating the subject with the mono-immunotherapy and a chemo-immunotherapy if the survival metric is below the threshold.

Embodiments of the present disclosure further comprise methods for selecting a treatment for a subject having a cancer. In one or more embodiments, the method comprises: receiving, using the one or more processors, one or more digital pathology features; inputting, using the one or more processors, the one or more digital pathology features into one or more statistical models; predicting, using the one or more processors, a survival metric indicative of a likelihood of survival of the individual, based on outputs of the one or more statistical models; selecting a mono-immunotherapy as the treatment if the survival metric is above a threshold; and selecting the mono-immunotherapy and a chemo-immunotherapy as the treatment if the survival metric is below the threshold.

In one or more embodiments, the method comprises: identifying one or more treatment options for a subject having a cancer. In one or more embodiments, the method comprises: receiving, using the one or more processors, one or more digital pathology features; inputting, using the one or more processors, the one or more digital pathology features into one or more statistical models; predicting, using the one or more processors, a survival metric indicative of a likelihood of survival of the individual, based on outputs of the one or more statistical models; identifying a mono-immunotherapy as the treatment if the survival metric is above a threshold; and identifying the mono-immunotherapy and a chemo-immunotherapy as the treatment if the survival metric is below the threshold.

Embodiments of the present disclosure further comprise methods for treating a subject having a cancer. In one or more embodiments, the method comprises: receiving, using one or more processors, sequence read data associated with a sample from the individual; selecting, using the one or more processors, a plurality of sequence reads from the sequence read data; determining, using the one or more processors, one or more characterization features based on the plurality of sequence reads; inputting, using the one or more processors, the one or more characterization features into one or more statistical models; predicting, using the one or more processors, a survival metric indicative of a likelihood of survival of the individual, based on outputs of the one or more statistical models; treating the subject with a mono-immunotherapy if the survival metric is above a threshold; and treating the subject with the mono-immunotherapy and a chemo-immunotherapy if the survival metric is below the threshold.

Embodiments of the present disclosure further comprise methods for selecting a treatment for a subject having a cancer. In one or more embodiments, the method comprises: receiving, using one or more processors, sequence read data associated with a sample from the individual; selecting, using the one or more processors, a plurality of sequence reads from the sequence read data; determining, using the one or more processors, one or more characterization features based on the plurality of sequence reads; inputting, using the one or more processors, the one or more characterization features into one or more statistical models; predicting, using the one or more processors, a survival metric indicative of a likelihood of survival of the individual, based on outputs of the one or more statistical models; selecting a mono-immunotherapy as the treatment if the survival metric is above a threshold; and selecting the mono-immunotherapy and a chemo-immunotherapy as the treatment if the survival metric is below the threshold.

Embodiments of the present disclosure further comprise methods for identifying one or more treatment options for a subject having a cancer. In one or more embodiments, the method comprises: receiving, using one or more processors, sequence read data associated with a sample from the individual; selecting, using the one or more processors, a plurality of sequence reads from the sequence read data; determining, using the one or more processors, one or more characterization features based on the plurality of sequence reads; receiving, using the one or more processors, one or more digital pathology features; inputting, using the one or more processors, the one or more characterization features and the one or more digital pathology features into one or more statistical models; predicting, using the one or more processors, a survival metric indicative of a likelihood of survival of the individual, based on outputs of the one or more statistical models; identifying a mono-immunotherapy as the treatment if the survival metric is above a threshold; and identifying the mono-immunotherapy and a chemo-immunotherapy as the treatment if the survival metric is below the threshold.

In one or more embodiments of the methods described above, the chemotherapy comprises one or more of a platinum agent and an antifolate agent. In one or more embodiments of the methods described above, the platinum agent is cisplatin or carboplatin. In one or more embodiments of the methods described above, the antifolate is pemetrexed. In one or more embodiments of the methods described above, the chemotherapy comprises cisplatin or carboplatin in combination with pemetrexed.

In one or more embodiments of the methods described above, the mono-immunotherapy comprises an immune checkpoint inhibitor (ICI). In one or more embodiments of the methods described above, the ICI comprises an inhibitor of PD-1 or PD-L1. In one or more embodiments of the methods described above, the cancer comprises lung cancer. In one or more embodiments of the methods described above, the individual has non-squamous non-small cell lung cancer (NSCLC) or advanced, non-squamous NSCLC.

In one or more embodiments of the methods described above, the one or more characterization features comprises a BCOR alteration, a BRAF alteration, a FAM123B alteration, a GATA3 alteration, a HGF alteration, an IRF2 alteration, a NF2 alteration, a NPM1 alteration, a PIK3CA alteration, a RAD54L alteration, a SDHC alteration, a SMAD4 alteration, and a tumor mutational burden. In one or more embodiments of the methods described above, the one or more digital pathology features comprises a granulocyte cell count over an immune cell count, a cluster dispersion standard deviation of lymphocytes in a cancer epithelium; a cluster size standard deviation of cancer cells in the cancer epithelium, a solidity of a largest region of a cancer stroma, and a total Euler number of the cancer epithelium.

Embodiments of the present disclosure further comprise methods for diagnosing a disease, the methods comprising: diagnosing that a subject has the disease based on a determination of a survival metric indicative of a likelihood of survival of the subject for a sample from the subject, wherein survival metric is determined according to the method of any one of claims 1 to 105.

Embodiments of the present disclosure further comprise methods for selecting an anti-cancer therapy, the methods comprising: responsive to determining a survival metric indicative of a likelihood of survival of the subject for a sample from a subject, selecting an anti-cancer therapy for the subject, wherein survival metric is determined according to the methods described above.

Embodiments of the present disclosure further comprise methods for treating a cancer in a subject, the methods comprising: responsive to determining survival metric indicative of a likelihood of survival of the subject for a sample from the subject, administering an effective amount of an anti-cancer therapy to the subject, wherein survival metric is determined according to the method of any of the methods described above.

Embodiments of the present disclosure further comprise methods for monitoring cancer progression or recurrence in a subject, the methods comprising: determining a first survival metric indicative of a likelihood of survival of the subject in a first sample obtained from the subject at a first time point according to the method of any of the methods described above; determining a second survival metric in a second sample obtained from the subject at a second time point; and comparing the first survival metric to the second survival metric, thereby monitoring the cancer progression or recurrence. In one or more embodiments, the second survival metric for the second sample is determined according to the method of any of the methods described above.

In one or more embodiments, the method can further comprise selecting an anti-cancer therapy for the subject in response to the cancer progression. In one or more embodiments, the method can further comprise administering an anti-cancer therapy to the subject in response to the cancer progression. In one or more embodiments, the method can further comprise adjusting an anti-cancer therapy for the subject in response to the cancer progression. In one or more embodiments, the method can further comprise adjusting a dosage of the anti-cancer therapy or selecting a different anti-cancer therapy in response to the cancer progression. In one or more embodiments, the method can further comprise comprising administering the adjusted anti-cancer therapy to the subject.

In one or more embodiments, the first time point is before the subject has been administered an anti-cancer therapy, and wherein the second time point is after the subject has been administered the anti-cancer therapy.

In one or more embodiments, the subject has a cancer, is at risk of having a cancer, is being routine tested for cancer, or is suspected of having a cancer. In one or more embodiments, the cancer is a solid tumor. In one or more embodiments, the cancer is a hematological cancer. In one or more embodiments, the anti-cancer therapy comprises chemotherapy, radiation therapy, immunotherapy, a targeted therapy, or surgery.

In one or more embodiments, the method further comprises determining, identifying, or applying the value of a survival metric indicative of a likelihood of survival of the subject for the sample as a diagnostic value associated with the sample. In one or more embodiments, the method further comprises generating a genomic profile for the subject based on the determination of a survival metric indicative of a likelihood of survival of the subject.

In one or more embodiments, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof. In one or more embodiments, the genomic profile for the subject further comprises results from a nucleic acid sequencing-based test.

In one or more embodiments, the method further comprises selecting an anti-cancer therapy, administering an anti-cancer therapy, or applying an anti-cancer therapy to the subject based on the generated genomic profile.

In one or more embodiments, the determination of a survival metric indicative of a likelihood of survival of the subject for the sample is used in making suggested treatment decisions for the subject. In one or more embodiments, the determination of a survival metric indicative of a likelihood of survival of the subject or the sample is used in applying or administering a treatment to the subject.

Embodiments of the present disclosure further comprise one or more systems. In some embodiments, the system can comprise: one or more processors; and a memory communicatively coupled to the one or more processors and configured to store instructions that, when executed by the one or more processors, cause the system to perform a method. In some embodiments, the instructions correspond to a method comprising: receiving, using the one or more processors, sequence read data associated with a sample from the individual; selecting, using the one or more processors, a plurality of sequence reads from the sequence read data; determining, using the one or more processors, one or more characterization features based on the plurality of sequence reads; receiving, using the one or more processors, one or more digital pathology features; inputting, using the one or more processors, the one or more characterization features and the one or more digital pathology features into one or more statistical models; and predicting, using the one or more processors, a survival metric indicative of a likelihood of survival of the individual, based on outputs of the one or more statistical models.

Embodiments of the present disclosure further comprise one or more non-transitory computer-readable storage mediums storing one or more programs, the one or more programs comprising instructions, which when executed by one or more processors of a system, cause the system to perform a method comprising: receiving, using the one or more processors, sequence read data associated with a sample from the individual; selecting, using the one or more processors, a plurality of sequence reads from the sequence read data; determining, using the one or more processors, one or more characterization features based on the plurality of sequence reads; receiving, using the one or more processors, one or more digital pathology features; inputting, using the one or more processors, the one or more characterization features and the one or more digital pathology features into one or more statistical models; and predicting, using the one or more processors, a survival metric indicative of a likelihood of survival of the individual, based on outputs of the one or more statistical models.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a process for predicting a survival metric based on a patient sample, in accordance with some embodiments of the present disclosure.

FIGS. 2A and 2B provide non-limiting examples of characterization features, in accordance with some embodiments of the present disclosure.

FIGS. 3A-3C provide non-limiting examples of quantitative pathology features, in accordance with some embodiments of the present disclosure.

FIG. 4 provides a non-limiting example of a block diagram associated with a process for predicting a risk score, in accordance with some embodiments of the present disclosure.

FIG. 5 provides a non-limiting example of a block diagram associated with a process for predicting a risk score, in accordance with some embodiments of the present disclosure.

FIG. 6 provides a non-limiting example of a block diagram associated with a process for predicting a survival metric, in accordance with some embodiments of the present disclosure.

FIG. 7 depicts an exemplary computing device or system in accordance with one embodiment of the present disclosure.

FIG. 8 depicts an exemplary computer system or computer network, in accordance with some instances of the systems described herein.

FIG. 9 provides a non-limiting example of data used to train a statistical model to predict a survival metric, in accordance with some embodiments of the present disclosure.

FIG. 10A provides a non-limiting example of a plot showing risk scores determined in accordance with some embodiments of the present disclosure.

FIGS. 10B and 10C provide non-limiting examples of plots showing survival metrics determined in accordance with some embodiments of the present disclosure.

FIGS. 10D and 10E provide non-limiting examples of plots showing hazard ratios determined in accordance with some embodiments of the present disclosure.

FIG. 11A provides a non-limiting example of a plot showing risk scores determined in accordance with some embodiments of the present disclosure.

FIGS. 11B and 11C provide non-limiting examples of plots showing survival metrics determined in accordance with some embodiments of the present disclosure.

FIGS. 11D and 11E provide non-limiting examples of plots showing hazard ratios determined in accordance with some embodiments of the present disclosure.

FIG. 12A provides a non-limiting example of a plot showing risk scores determined in accordance with some embodiments of the present disclosure.

FIGS. 12B and 12C provide non-limiting examples of plots showing survival metrics determined in accordance with some embodiments of the present disclosure.

FIGS. 12D and 12E provide non-limiting examples of plots showing hazard ratios determined in accordance with some embodiments of the present disclosure.

FIG. 13 provides a non-limiting example of a plots showing the concordance index for predictions by statistical models built in accordance with some embodiments of the present disclosure.

### DETAILED DESCRIPTION

Disclosed herein are methods and systems for determining a survival metric indicative of survival of a patient when treated with either mono-immunotherapy or chemo-immunotherapy. Embodiments of the present disclosure integrate information about the tissue micro-environment (e.g., corresponding to digital pathology images and/or associated quantitative pathology features (QPFs)) with genomic information (e.g., determined using next-generation sequencing (NGS) and clinical information. In some embodiments the survival metric may correspond to a biomarker that may aid healthcare providers in determining first line treatment decisions of whether to treat a patient with mono-immunotherapy or chemo-immunotherapy. Embodiments of the present disclosure may be applicable to individuals with advanced non-squamous non-small cell lung cancer (NSCLC) patients without actionable alterations in EGFR and ALK. A skilled artisan will understand that the systems and methods described herein may be applied to other types of cancer without departing from the scope of this disclosure.

Embodiments of the present disclosure may also provide systems and methods for providing bias correction regarding determining a patient's estimated overall survival. For instance, there is often a bias based on whether the sample is a biopsy or resection. For example, patients associated with needle core biopsy samples versus tumor resection samples tend to have worst overall survival. The sample type appears to be associated with the overall health of the patient and the extent of their metastatic disease at the time of surgery/sample biopsy. Embodiments of the present disclosure further provide bias correction based on the sample type.

Embodiments of the present disclosure further comprise methods for predicting a response to treatment or a survival of an individual with a disease. In one or more embodiments, the method comprises: receiving, using one or more processors, sequence read data associated with a sample from the individual; selecting, using the one or more processors, a plurality of sequence reads from the sequence read data; determining, using the one or more processors, one or more characterization features based on the plurality of sequence reads; receiving, using the one or more processors, one or more digital pathology features; inputting, using the one or more processors, the one or more characterization features and the one or more digital pathology features into one or more statistical models; and predicting, using the one or more processors, a survival metric indicative of a likelihood of survival of the individual or an estimated response to treatment, based on outputs of the one or more statistical models.

Embodiments of the present disclosure further comprise methods for predicting a response to treatment of an individual with a disease. In one or more embodiments, the method comprises: receiving, using one or more processors, sequence read data associated with a sample from the individual; selecting, using the one or more processors, a plurality of sequence reads from the sequence read data; determining, using the one or more processors, one or more characterization features based on the plurality of sequence reads; receiving, using the one or more processors, one or more digital pathology features; inputting, using the one or more processors, the one or more characterization features and the one or more digital pathology features into one or more statistical models; and predicting, using the one or more processors, a survival metric indicative of an estimated response of the individual to the treatment, based on outputs of the one or more statistical models.

In one or more embodiments, the one or more characterization features comprises: a BCOR alteration, a BRAF alteration, a FAM123B alteration, a GATA3 alteration, a HGF alteration, an IRF2 alteration, a NF2 alteration, a NPM1 alteration, a PIK3CA alteration, a RAD54L alteration, a SDHC alteration, a SMAD4 alteration, and a tumor mutational burden, and wherein the one or more digital pathology features comprises a granulocyte cell count over an immune cell count, a cluster dispersion standard deviation of lymphocytes in a cancer epithelium, a cluster size standard deviation of cancer cells in the cancer epithelium, a solidity of a largest region of a cancer stroma, and a total Euler number of the cancer epithelium.

In one or more embodiments, the one or more characterization features comprise a presence of a genomic alteration, an absence of a genomic alteration, a tumor mutational burden (TMB), a PD-L1 status, chromosome arm level, cytoband level copy number alterations, mutational signatures, copy number signatures, clinical features or a combination thereof. In one or more embodiments, the PD-L1 status comprises a negative status, a positive-low status, or a positive-high status. In one or more embodiments, the genomic alteration comprises a single nucleotide variant (SNV), insertion and/or deletion (indel), rearrangement, or copy number variant (CNV). In one or more embodiments, the one or more clinical features comprises the sample type and the method further comprises pre-processing the one or more clinical features to account for bias associated with the sample type.

In one or more embodiments, one or more digital pathology features comprise a total cancer epithelium value, a regional cancer epithelium value, an area of a largest region of cancer epithelium, a perimeter of the largest region of cancer epithelium, a filled area of the largest region of cancer epithelium, a number of clusters of cells in the cancer epithelium, or a combination thereof.

In one or more embodiments, the method further comprises training the statistical model, wherein training the statistical model comprises: receiving, using the one or more processors, training data based on a plurality of training samples; and training, using the one or more processors, the statistical model based on the training data to obtain a trained statistical model.

In one or more embodiments, training the statistical model comprises: inputting, using the one or more processors, the training data into the statistical model; determining, using the one or more processors, a score based on the training data; and updating, using the one or more processors, one or more weights associated with the statistical model based on the score. In one or more embodiments, the one or more weights are associated with a specific disease. In one or more embodiments, the method further comprises filtering, using the one or more processors, the training data to select the one or more characterization features and the one or more digital pathology features for the trained statistical model based on the weights. In one or more embodiments, the training data comprises one or more training characterization features, one or more training digital pathology features, one or more training clinical features, one or more clinical outcomes, or a combination thereof.

In one or more embodiments, the method further comprises assigning, using the one or more processors, a therapy for the individual based on the predicted survival metric. In one or more embodiments, the method further comprises administering, using the one or more processors, a treatment to the individual based on the predicted survival metric. In one or more embodiments, the method further comprises associating, using the one or more processors, the individual with a clinical trial based on the predicted survival metric. In one or more embodiments, the method further comprises monitoring, using the one or more processors, a prognosis of the individual based on the predicted survival metric. In one or more embodiments, the method further comprises predicting, using the one or more processors, one or more clinical outcomes based on the predicted survival metric.

Accordingly, embodiments of the present disclosure provide systems and methods for training and using a statistical model with characterization features and/or digital pathology features (e.g., QPFs). In one or more embodiments, combining the predictive power of the characterization features and the quantitative features provide a more accurate way to predict the survival of an individual compared to survival predictions based on these features alone. These survival predictions may be used by the system to make treatment and therapy recommendations to improve survival outcomes for patients.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

The term “quantitative pathology feature” (QPF) refers to a numerical assessment of a region of interest in a pathology image, which may be the region's area (e.g. area of cancer cells), a count of cells or structures (e.g. number of lymphocytes in a region), or another countable quantity.

The section headings used herein are for organizational purposes only and are not to be construed as limiting the subject matter described.

### Methods for Predicting an Individual's Survival

Disclosed herein are methods and systems for determining a survival metric indicative of survival of a patient when treated with either mono-immunotherapy or combination mono-immunotherapy and chemo-immunotherapy. Embodiments of the present disclosure integrate information about the tissue micro-environment (e.g., corresponding to digital pathology images and/or associated quantitative pathology features (QPFs)) with genomic information (e.g., determined using next-generation sequencing (NGS) and clinical information. In some embodiments the survival metric can be a biomarker that may aid healthcare providers in determining first line treatment decisions of whether to treat a patient with mono-immunotherapy or chemo-immunotherapy.

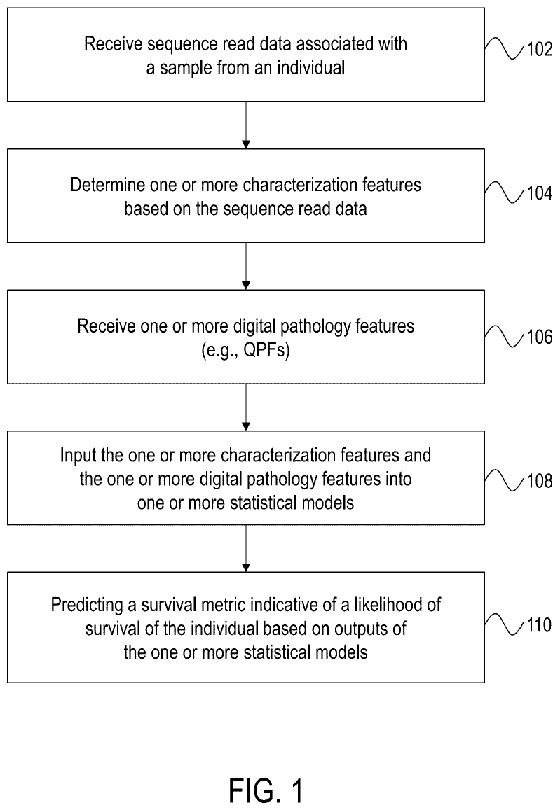

FIG. 1 provides a non-limiting example of a process 100 for predicting an individual's response to an immunotherapy treatment, such as mono-immunotherapy treatment or chemo-immunotherapy. Process 100 can be performed, for example, using one or more electronic devices implementing a software platform. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

At block 102 of FIG. 1 the system can receive sequence read data associated with a sample from an individual. In one or more examples, the sample may be a solid biopsy sample such as a tissue resection or a core needle biopsy. In some examples, other types of tissue samples may be utilized including but not limited to fine needle aspirate, pleural efflux, blood smear, bone marrow smear, and the like. In some instances, the sequence read data may be derived from single region sequencing (e.g., sequencing of a single tissue biopsy sample collected from the tumor of the individual). In some instances, the genomic data comprising sequence read data may be derived from multi-region sequencing (e.g., sequencing of multiple tissue biopsy samples collected from the tumor of the individual). In some instances, the genomic data comprising sequence read data may be derived from single cell sequencing data as opposed to bulk tumor sequencing.

In some instances, the genomic data comprising sequence read data may be derived from targeted sequencing, e.g., targeted exome sequencing. In some instances, the genomic data comprising sequence read data may be derived from broad panel sequencing. In some instances, the sequence read data may be derived from whole genome or whole exome sequencing, e.g., as opposed to targeted exome sequencing or broad panel sequencing to increase the number of genomic features (e.g., the number of short variants, copy number alteration) detected. In one or more examples, the sequence read data may be received by the system as a BAM file.

At block 104 of FIG. 1, the system can determine a one or more characterization features of the sample based on the sequence read data. Due to the computational complexity involved in processing the sequence read data, the system can use one or more processors to determine one or more characterization features of the sample. In one or more examples, the sequence read data may be indicative of a presence or absence of one or more short variants (SVs) in a patient sample. The sequence read data may also be indicative of the presence or absence of characterizing events, such as copy number signatures, rearrangements, insertions, deletions, fusions, chromosomal aneuploidy, whole genome doubling, PD-L1 status, cytoband level copy number alterations, mutational signatures (e.g., Catalogue Of Somatic Mutations In Cancer (COSMIC) mutational signatures), microsatellite instability (MSI) status, tumor mutational burden (TMB), PD-L1 status, or any combination thereof.

The TMB may correspond to a value indicative of the number of mutations found in the sample. The PD-L1 status can be categorized as negative, positive-low, positive-high based on the proportion of tumor cells that stain for PD-L1 through immunohistochemistry. The PD-L1 status can be used as a biomarker for selecting patients who will benefit from immune checkpoint inhibitors. The mutational signatures may correspond to a indication of whether a particular mutational signature is present in the sample. The MSI status may be categorized as high, unknown, stable, and the like. The MSI status may be indicative of the number of mutations within microsatellites.

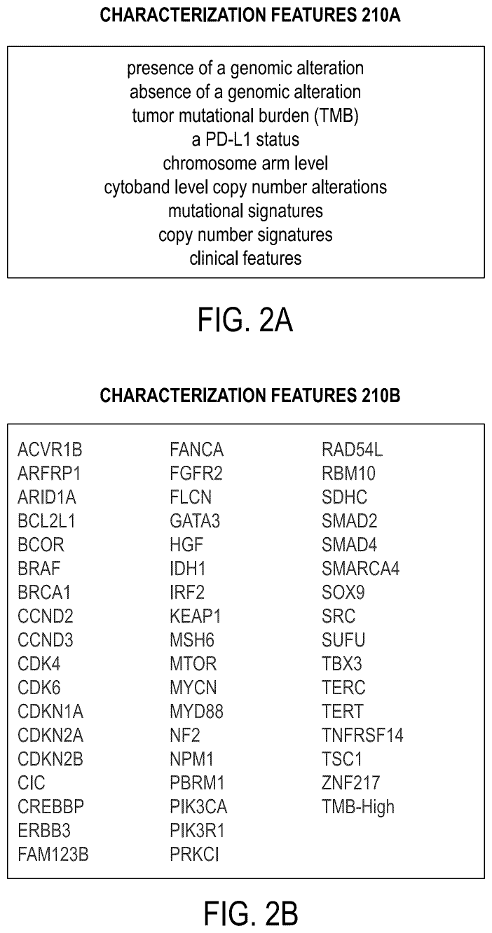

As shown in FIG. 2A, the characterization features 210A may include, for example, a presence of a genomic alteration, an absence of a genomic alteration, a TMB status, a PD-L1 status, a chromosome arm level, cytoband level copy number alterations, mutational signatures, copy number signatures, clinical features, or any combination thereof. The genomic alteration can be associated with one or more of a single nucleotide variant (SNV), insertion and/or deletion (indel), repetitive element (rearrangement), or copy number variant (CNV). In some instances, the clinical features can include, for example information regarding the patient (e.g., age, weight, sex, race/ethnicity, smoking history, stage, histologic subtype, ECOG performance status, etc.), information regarding the sample (e.g., sample-type), and the like.

As a non-limiting example, the characterization features 210B may include, but is not limited to ACVR1B, ARFRP1, ARID1A, BCL2L1, BCOR, BRAF, BRCA1, CCND2, CCND3, CDK4, CDK6, CDKN1A, CDKN2A, CDKN2B, CIC, CREBBP, ERBB3, FAM123B, FANCA, FGFR2, FLCN, GATA3, HGF, IDH1, IRF2, KEAP1, MSH6, MTOR, MYCN, MYD88, NF2, NPM1, PBRM1, PIK3CA, PIK3R1, PRKC1, RAD54L, RBM10, SDHC, SMAD2, SMAD4, SMARCA4, SOX9, SRC, SUFU, TBX3, TERC, TERT, TNFRSF14, TSC1, ZNF217, TMB-High, or any combination thereof. For example, in some embodiments, the characterization features 210B may refer to sequence information, mutational signature(s), and/or genomic alteration(s) (e.g., SNV, indel, rearrangement, CNV, etc.) associated with one or more of the ACVR1B, ARFRP1, ARID1A, BCL2L1, BCOR, BRAF, BRCA1, CCND2, CCND3, CDK4, CDK6, CDKN1A, CDKN2A, CDKN2B, CIC, CREBBP, ERBB3, FAM123B, FANCA, FGFR2, FLCN, GATA3, HGF, IDH1, IRF2, KEAP1, MSH6, MTOR, MYCN, MYD88, NF2, NPM1, PBRM1, PIK3CA, PIK3R1, PRKC1, RAD54L, RBM10, SDHC, SMAD2, SMAD4, SMARCA4, SOX9, SRC, SUFU, TBX3, TERC, TERT, TNFRSF14, TSC1, and ZNF217 genes, or any combination thereof. In some embodiments, ACVR1B, ARFRP1, ARID1A, BCL2L1, BCOR, BRAF, BRCA1, CCND2, CCND3, CDK4, CDK6, CDKN1A, CDKN2A, CDKN2B, CIC, CREBBP, ERBB3, FAM123B, FANCA, FGFR2, FLCN, GATA3, HGF, IDH1, IRF2, KEAP1, MSH6, MTOR, MYCN, MYD88, NF2, NPM1, PBRM1, PIK3CA, PIK3R1, PRKC1, RAD54L, RBM10, SDHC, SMAD2, SMAD4, SMARCA4, SOX9, SRC, SUFU, TBX3, TERC, TERT, TNFRSF14, TSC1, and ZNF217 refer to the corresponding human gene.

At block 106 of FIG. 1, the system can receive one or more digital pathology features. In some instances, the digital pathology features may be generated by a machine learning platform based on histopathology slides or images. The machine learning platform may include one or more machine learning models (e.g., a convolutional neural network (CNN)) that is trained to provide quantitative pathology features (QPFs) that are interpretable, quantitative summaries of the tumor and tumor microenvironment based on a pathology image. In some examples, the machine learning platform can output a feature vector of human-interpretable features (HIFs) extracted from a corresponding pathology image. The HIFs may include simple quantified descriptions of cells or tissue regions (e.g., density of lymphocytes in cancer tissue) to more complex multi-component spatial relationships. The machine learning platform is described in more detail in, e.g., U.S. Pat. No. 10,650,520, the entire contents of which are incorporated herein by reference.

In some instances, the digital pathology features 320A can include, but are not limited to, cell-type counts and densities across different tissue regions (e.g., density of plasma cells in cancer tissue); cell-level cluster features that capture inter-cellular spatial relationships (e.g., cluster dispersion, size, and extent); cell-level proportion and proximity features (e.g., a proportional count of lymphocytes versus fibroblasts within 80 microns (μm) of the cancer-stroma interface); tissue area and multiplicity counts (e.g., number of significant regions of cancer tissue); tissue architecture features (e.g., the average solidity (solidness) of cancer tissue regions or the fractal dimension (geometrical complexity) of CAS); tissue-level morphology features (e.g., Euler numbers, perimeter over area (shape roughness), lacunarity (gappiness), and eccentricity); size-based measures (e.g., number of connected components, major axis length, minor axis length, convex areas, filled areas). Additionally, details regarding HIFs may be found in Diao, J. A., Wang, J. K., Chui, W. F. et al. Human-interpretable image features derived from densely mapped cancer pathology slides predict diverse molecular phenotypes. Nat Commun, 1613 (2021), the entire contents of which are incorporated herein by reference.

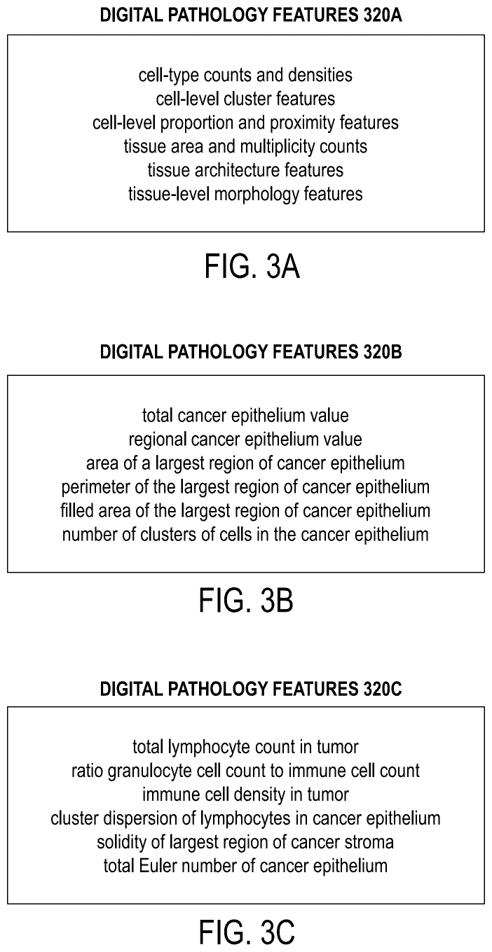

As shown in FIG. 3B, in some instances, the digital pathology features 320B can include, but are not limited to a total cancer epithelium value, a regional cancer epithelium value, an area of a largest region of cancer epithelium, a perimeter of the largest region of cancer epithelium, a filled area of the largest region of cancer epithelium, a number of clusters of cells in the cancer epithelium, or a combination thereof.

As shown in FIG. 3C, in some examples, the digital pathology features 320C can include but are not limited to a total lymphocyte count in tumor, a ratio granulocyte cell count to immune cell count, a immune cell density in tumor, a cluster dispersion of lymphocytes in cancer epithelium, a solidity of largest region of cancer stroma, a total Euler number of cancer epithelium, or any combination thereof.

At block 108 of FIG. 1, the system can input the one or more characterization features and the one or more digital pathology features into one or more statistical models. In some embodiments, the system may input the characterizing features and/or the QPFs. In one or more embodiments, the one or more characterization features and the one or more digital pathology features may include the features shown in Table I, provided below.

TABLE I
Feature

[[GRANULOCYTE CELLS] OVER [IMMUNE CELLS]] IN [TUMOR]_HE
CLUSTER DISPERSION SD LYMPHOCYTE IN CANCER EPITHELIUM
(UNVALIDATED)_HE
CLUSTER SIZE STANDARD DEVIATION CANCER CELL IN CANCER
EPITHELIUM (UNVALIDATED)_HE
SOLIDITY OF LARGEST REGION OF CANCER STROMA_HE
TOTAL EULER NUMBER OF CANCER EPITHELIUM_HE
BCOR
BRAF
FAM123B
GATA3
HGF
IRF2
NF2
NPM1
PIK3CA
RAD54L
SDHC
SMAD4
tmb

The one or more statistical models may be trained to determine a risk score or survival score indicative of a likelihood of survival of the individual based on the inputs. The risk score may be used to predict a likelihood of survival and to make treatment decisions for the individual. In one or more examples, the statistical model can be a trained machine learning model. In one or more examples, the trained machine learning model may be a classifier model, for example, a random forest model or a random forest survival model. In one or more embodiments, the trained machine learning model may be a regression model, for example, a LASSO regression model (e.g., LASSO Cox regression model).

In one or more examples, the statistical model may be part of a machine learning process. In one or more examples, the machine learning model can include an artificial intelligence (“AI”) learning model. In some instances, the machine learning model can be at least one of a supervised model or an unsupervised model. In one or more examples, the machine learning model can include one or more machine learning models, such as an extreme gradient boosting model, a logistic regression model, a LASSO regression model (e.g., LASSO Cox regression model), a penalized Cox regression model, an elastic net model, a ridge regression model, a random forest model (e.g., survival random forest model), a support vector machine model, a k-nearest neighbor model, a Bayesian model, a naïve-based model, a Gaussian naïve-based model, a clustering model, a limited expectation maximization model, a gradient boosting ensemble model, an adaboost model, a bagging model, a neural network model, a backpropagation model, a stochastic gradient descent model, a linear regression model, a non-linear regression model, a multivariate regression model, a robust machine learning model, a survival tree model, a discrete-time survival model, and a proportional hazards model.

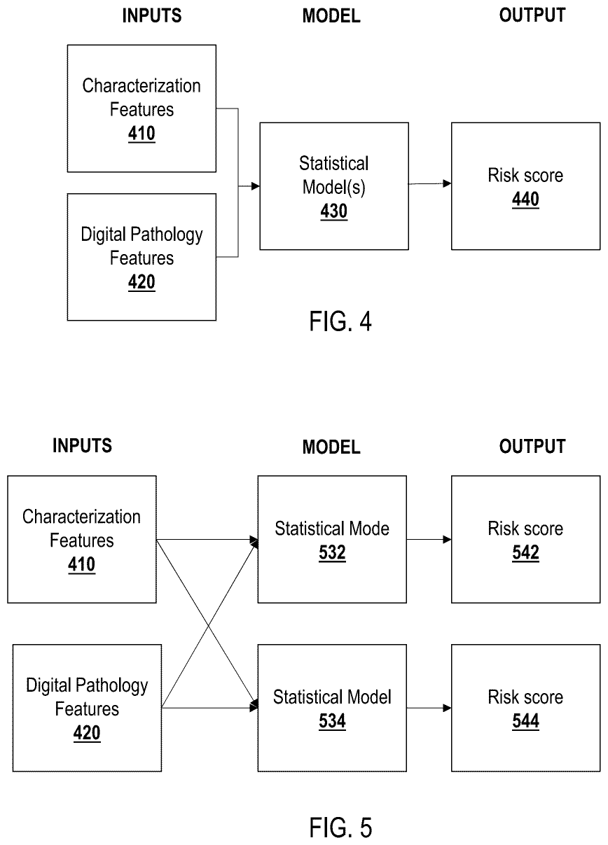

FIG. 4 illustrates an exemplary block diagram 400 corresponding to a process for determining a risk score that can be used to determine a survival metric indicative of a likelihood of survival of an individual. As shown in the figure, the statistical model(s) 430 can receive one or more characterization features 410 and one or more digital pathology features 420 as inputs. In some embodiments, the model(s) 430 may be configured to receive characterization features 410 or digital pathology features 420 (e.g., QPFs). The model(s) 430 can be prediction model(s) configured to determine a risk score 440 as an output. In some embodiments, the risk score may be used to determine a survival metric that is indicative of a prediction of a likelihood of survival of the individual.

FIG. 5 illustrates an exemplary block diagram 500 corresponding to a process for determining a risk score indicative of a likelihood of survival of an individual. As shown in the figure, the statistical models 532 and 534 can receive one or more characterization features 510 and one or more digital pathology features 520 as inputs. The statistical models 532 and 534 can be prediction models configured to determine respective risk scores. In some embodiments, the risk score may be used to determine a survival metric that is indicative of a prediction of a likelihood of survival of the individual. In some examples, the statistical model 532 may be trained to provide a risk score used to determine the survival metric 542 of an individual when treated with a mono-immunotherapy and the statistical model 534 may be trained to provide a risk score used to determine the survival metric 544 of an individual when treated with a chemo-immunotherapy.

Returning to FIG. 1, at block 110, the system can determine a survival metric indicative of a likelihood of survival of the individual based on outputs (e.g., risk scores) of the one or more statistical models. In some embodiments, the survival metric may be determined based on a risk score output by the statistical model. Because the one or more statistical models are trained on the likelihood of survival, the output of the one or more statistical models is associated with a patient's risk. In some embodiments, a threshold may be selected to stratify patients into one or more groups associated with a risk substantially higher than average and/or substantially below average. The threshold may be selected to optimize a particular test statistic. For example, threshold may be selected to optimize a risk score value that results in the largest hazard ratio between the high and low risk group.

In some embodiments, different survival metrics may be associated with different types of treatment. That is, different survival metrics associated with different treatment types (e.g., immunotherapy, mono-immunotherapy, chemo-immunotherapy, and combination therapy (e.g., mono-immunotherapy and chemo-immunotherapy)) may be determined based on the risk score. In some examples the risk score may be compared to one or more thresholds to determine the survival metric. For instance, patients with a risk score that is above the threshold may be associated with a poor likelihood of survival while patients with a risk score below the threshold may be associated with a strong likelihood of survival. In some embodiments, different thresholds may be associated with different treatment types.

In some embodiments, the one or more thresholds may be selected based on results of the training data. For instance, in some examples, a threshold may be selected such that 50% of the data is above the threshold and 50% of the data is below the threshold. In some embodiments, there may be two or more thresholds. For instance, there may be a first threshold and a second threshold such that risk scores determined based on the training data are divided into tertiles. In some embodiments, the thresholds may be selected to divide the data using, for example, an F1-score, an F2-score, Mathew's correlation coefficient, Newton's index, a time-dependent ROC curve, a hazard ratio and p-value, a likelihood ratio test, a concordance index (C-index), or a combination thereof.

In some examples, the risk score(s) output by the statistical model may be used to classify patients based on their likelihood of survival, e.g., poor survival or better survival. As discussed above, the risk score may be associated with different survival metrics corresponding to different treatment types. In some embodiments, the survival metric may correspond to a percentage of a likelihood of survival of the individual. For instance, a poor likelihood of survival may be associated with a survival of less than 50% for a predetermined period of time.

In some embodiments, the survival metric may be used to predict the likelihood of a patient's survival over a predetermined time period. For example, the survival metric may correspond to an individual's predicted six-month survival overall survival or predicted six-month progression-free survival. In some instances, the survival metric may be associated with a particular treatment. For example, the survival metric may be indicative of a patient's likelihood of survival when treated with a mono-immunotherapy. In some examples, the survival metric may be indicative of a patient's likelihood of survival when treated with a combination therapy. These survival metrics are exemplary and other survival metrics and/or other time periods may be used without departing from the scope of this disclosure.

In some embodiments, the system may provide a first line treatment recommendation based on the survival metric. For instance, the survival metric may be associated with a treatment comprising immunotherapy such as mono-immunotherapy or mono-immunotherapy in combination with chemo-immunotherapy. In some examples, if the survival metric for treatment of the individual with a mono-immunotherapy indicates a better likelihood of survival than treatment of the individual with a combination therapy, then the system may recommend that the healthcare providers treat the patient with a mono-immunotherapy. As another example, if the survival metric for treatment of the individual with a mono-immunotherapy indicates a lower likelihood of survival than treatment of the individual with a chemo-immunotherapy, then the system may recommend that the healthcare providers treat the patient with the combination mono-immunotherapy and chemo-immunotherapy.

In some embodiments, the chemo-immunotherapy can include one or more of a platinum agent (e.g., cisplatin or carboplatin) and an antifolate agent (e.g., pemetrexed). In some examples, the combination mono-immunotherapy and chemo-immunotherapy can include cisplatin or carboplatin in combination with pemetrexed.

In some examples, the immunotherapy can comprise an immune checkpoint inhibitor (ICI). In some embodiments, the ICI targets PD-L1, PD-1, CTLA-4, CEACAM, LAIR1, CD160, 2B4, CD80, CD86, CD276, VTCN1, HVEM, KIR, A2AR, MHC class I, MHC class II, GALS, adenosine, TGFR, OX40, CD137, CD40, IDO, CSFIR, TIM-3, BTLA, VISTA, LAG-3, TIGIT, IDO, MICA/B, and/or arginase.

In some examples, the ICI can comprise an inhibitor of PD-1 or PD-L1. In some embodiments, the agent that inhibits PD-1 is a small molecule, a nucleic acid, a polypeptide, carbohydrate, a lipid, a metal, or a toxin. In some embodiments, the agent that inhibits PD-1 is a PD-1 binding antagonist. In some embodiments, the PD-1 binding antagonist is an antibody, antibody-drug conjugate, antibody fragment, or immunoadhesin. In some embodiments, the PD-1 binding antagonist is selected from the group consisting of pembrolizumab, nivolumab, cemiplimab, spartalizumab, genolimzumab, SHR1210, JS001, BGB-108, BGB-A317, IBI308, GLS-010, BMS-936558, BCD-100, REGN2810, MGA-012, BI 754091, STI-A1110, INCSHR-1210, PF-06801591, TSR-042, AM0001, JNJ-63723283, and ENUM 244C8. In some embodiments, the checkpoint inhibitor is an agent that inhibits PD-L1 and/or PD-L2. In some embodiments, the agent that inhibits PD-L1 and/or PD-L2 is a small molecule, a nucleic acid, a polypeptide, carbohydrate, a lipid, a metal, or a toxin. In some embodiments, the agent that inhibits PD-L1 is a PD-L1 binding antagonist. In some embodiments, the PD-L1 binding antagonist is an antibody, antibody-drug conjugate, antibody fragment, or immunoadhesin. In some embodiments, the PD-L1 binding antagonist is selected from the group consisting of atezolizumab, avelumab, durvalumab, KN035, CS1001, MDX-1105, LY3300054, STI-A1014, FAZ053, and CX-072. In some embodiments, the checkpoint inhibitor is an agent that inhibits CTLA4. In some embodiments, the agent that inhibits CTLA4 is a small molecule, a nucleic acid, a polypeptide, carbohydrate, a lipid, a metal, or a toxin. In some embodiments, the agent that inhibits CTLA4 is an antibody, antibody-drug conjugate, antibody fragment, or immunoadhesin. In some embodiments, the agent that inhibits CTLA4 is selected from the group consisting of ipilimumab, APL-509, AGEN1884, and CS1002.

In some embodiments, the immunotherapy can comprise one or more of a cancer vaccine, cell-based therapy, T cell receptor (TCR)-based therapy, adjuvant immunotherapy, cytokine immunotherapy, or oncolytic virus therapy. Examples of immunotherapies are described in greater detail infra but are not intended to be limiting.

In some embodiments, the immunotherapy comprises a cancer vaccine. A range of cancer vaccines have been tested that employ different approaches to promoting an immune response against the tumor (see, e.g., Emens L A, Expert Opin Emerg Drugs 13(2): 295-308 (2008) and US20190367613). Approaches have been designed to enhance the response of B cells, T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello, A. et ah, J. Clin. Invest. 95:341, 1995), naked cDNA or peptides, encapsulated e.g., in poly(DL-lactide-co-glycolide) (“PLG”) microspheres (see, e.g., Eldridge, et ah, Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tam, J. P., J. Immunol. Methods 196:17-32, 1996). In some embodiments, a cancer vaccine is formulated as a peptide-based vaccine, or nucleic acid based vaccine in which the nucleic acid encodes the polypeptides. In some embodiments, a cancer vaccine is formulated as an antibody based vaccine. In some embodiments, a cancer vaccine is formulated as a cell based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al, Cancer Sci, 104:14-21), 2013). In some embodiments, such cancer vaccines augment the anti-tumor response.

In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (Pexa Vec/JX-594, SillaJen/formerly Jennerex Biotherapeutics), a thymidine kinase-(TK−) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818); anti-gp 100; STINGVAX; GVAX; DCVaxL; and DNX-2401. In some embodiments, the cancer vaccine is selected from JX-929 (SillaJen/formerly Jennerex Biotherapeutics), a TK− and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (Vira Therapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into the patient (possibly with proinflammatory or other attractants such as GM-CSF), taken up by cells in vivo to make the specific antigens, which would then provoke the desired immune response. In some embodiments, vectors may be used to deliver more than one tumor antigen at a time, to increase the immune response. In addition, recombinant virus, bacteria or yeast vectors should trigger their own immune responses, which may also enhance the overall immune response.

In some embodiments, the cancer vaccine comprises a DNA-based vaccine. In some embodiments, DNA-based vaccines can be employed to stimulate an anti-tumor response. The ability of directly injected DNA, that encodes an antigenic protein, to elicit a protective immune response has been demonstrated in numerous experimental systems. Vaccination through directly injecting DNA, that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12:771-776). In some embodiments, plasmid (or other vector) DNA that includes a sequence encoding a protein operably linked to regulatory elements required for gene expression is administered to individuals (e.g. human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide (i.e. comprising HPV antigens), or may be transcribed to provide further transcripts with the same sense as the delivered RNA which are translated to provide in situ expression of the antigen.

In some embodiments, the immunotherapy comprises a cell-based therapy. In some embodiments, the immunotherapy comprises a T cell-based therapy. In some embodiments, the immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) tumor cells. In some embodiments, the immune response results in inhibition of tumor and/or metastatic cell growth and/or proliferation and in related embodiments results in neoplastic cell death and/or resorption. The immune cells can be derived from a different organism/host (exogenous immune cells) or can be cells obtained from the subject organism (autologous immune cells). In some embodiments the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g. chimeric). In some embodiments, the population of immune cells can be obtained from a subject in need of therapy or suffering from a disease associated with reduced immune cell activity. Thus, the cells will be autologous to the subject in need of therapy. In some embodiments, the population of immune cells can be obtained from a donor, such as a histocompatibility matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy. Several basic approaches for the derivation, activation and expansion of functional anti-tumor effector cells have been described in the last two decades. These include: autologous cells, such as tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. In some embodiments, the T cells are derived from the blood, bone marrow, lymph, umbilical cord, or lymphoid organs. In some aspects, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation. In some embodiments, the sub-types and subpopulations of T cells (e.g., CD4⁺ and/or CD8⁺ T cells) are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In some embodiments, one or more of the T cell populations is enriched for or depleted of cells that are positive for a specific marker, such as surface markers, or that are negative for a specific marker. In some embodiments, such markers are those that are absent or expressed at relatively low levels on certain populations of T cells (e.g., non-memory cells) but are present or expressed at relatively higher levels on certain other populations of T cells (e.g., memory cells). In some embodiments, T cells are separated from a PBMC sample by negative selection of markers expressed on non-T cells, such as B cells, monocytes, or other white blood cells, such as CD 14. In some embodiments, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations. In some embodiments, CD8⁺ T cells are further enriched for or depleted of naive, central memory, effector memory, and/or central memory stem cells, such as by positive or negative selection based on surface antigens associated with the respective subpopulation. In some embodiments, the T cells are autologous T cells. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests are cultured in interleukin-2 (IL-2). The cells are cultured until confluence (e.g., about 2×10⁶ lymphocytes), e.g., from about 5 to about 21 days, such as from about 10 to about 14 days.

In some embodiments, the cultured T cells can be pooled and rapidly expanded. Rapid expansion provides an increase in the number of antigen-specific T-cells, e.g., of at least about 50-fold (e.g., 50-, 60-, 70-, 80-, 90-, or 100-fold, or greater) over a period of about 10 to about 14 days. In some embodiments, expansion can be accomplished by any of a number of methods as are known in the art. For example, T cells can be rapidly expanded using non-specific T-cell receptor stimulation in the presence of feeder lymphocytes and either interleukin-2 (IL-2) or interleukin-15 (IL-15), with IL-2 being particularly contemplated. The non-specific T-cell receptor stimulus can include around 30 ng/ml of OKT3, a mouse monoclonal anti-CD3 antibody (available from Ortho-McNeil®, Raritan, N.J.). In some embodiments, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as a human leukocyte antigen A2 (HLA-A2) binding peptide, in the presence of a T-cell growth factor, such as 300 IU/ml IL-2 or IL-15, with IL-2 being contemplated. The in vv/ro-induced T-cells are rapidly expanded by re stimulation with the same antigen(s) of the cancer pulsed onto HLA-A2-expressing antigen-presenting cells. In some embodiments, the T cells can be re-stimulated with irradiated, autologous lymphocytes or with irradiated HLA-A2+ allogeneic lymphocytes and IL-2, for example. In some embodiments, the autologous T-cells can be modified to express a T-cell growth factor that promotes the growth and activation of the autologous T-cells. In some embodiments, suitable T-cell growth factors include, for example, interleukin (IL)-2, IL-7, IL-15, and IL-12. Suitable methods of modification are known in the art. See, for instance, Sambrook et al, MOLECULAR CLONING: A LABORATORY MANUAL, 3^{rd} ed., Cold Spring Harbor Press, Cold Spring Harbor, N.Y. 2001; and Ausubel et al, CURRENT PROTOCOLS IN MOLECULAR BIOLOGY, Greene Publishing Associates and John Wiley & Sons, NY, 1994. In some embodiments, modified autologous T-cells express the T-cell growth factor at high levels. T-cell growth factor coding sequences, such as that of IL-12, are readily available in the art, as are promoters, the operable linkage of which to a T-cell growth factor coding sequence promote high-level expression. In some embodiments, autologous T cells may be engineered to express a defined T cell receptor (TCR) that are directed against target TAAs, either wild-type TCR, or mutated/engineered TCR towards a higher affinity to the antigen peptide/MHC molecule complexes. In some embodiments, autologous T cells may be engineered to express a CAR, e.g., as described infra.

In some embodiments, the T cell-based therapy comprises a chimeric antigen receptor (CAR)-T-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen. In some embodiments, the CAR specifically binds a neoantigen.

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen. In some embodiments, the recombinant TCR specifically binds a neoantigen.

In some embodiments, the T cell-based therapy comprises tumor-infiltrating lymphocytes (TILs). For example, TILs can be isolated from a tumor or cancer of the present disclosure, then isolated and expanded in vitro. In some embodiments, the TILs are exposed to one or more neoantigens of the present disclosure in vitro after isolation. TILs are then administered to the patient (optionally in combination with one or more cytokines or other immune-stimulating substances).

In some embodiments, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells constitute about 10% of the lymphocytes in human peripheral blood. When lymphocytes are cultured in the presence of interleukin 2 (IL-2), strong cytotoxic reactivity develops. NK cells are effector cells known as large granular lymphocytes because of their larger size and the presence of characteristic azurophilic granules in their cytoplasm. NK cells differentiate and mature in the bone marrow, lymph nodes, spleen, tonsils, and thymus. NK cells can be detected by specific surface markers, such as CD 16, CD56, and CD8 in humans. NK cells do not express T-cell antigen receptors, the pan T marker CD3, or surface immunoglobulin B cell receptors. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art. In some embodiments, umbilical CB is used to derive NK cells. In some embodiments, the NK cells are isolated and expanded by the previously described method of ex vivo expansion of NK cells (Spanholtz et al, 2011; Shah et al, 2013). In some embodiments, CB mononuclear cells are isolated by ficoll density gradient centrifugation and cultured in a bioreactor with IL-2 and artificial antigen presenting cells (aAPCs). After 7 days, the cell culture is depleted of any cells expressing CD3 and re-cultured for an additional 7 days. The cells are again CD3-depleted and characterized to determine the percentage of CD56/CD3 cells or NK cells. In some embodiments, umbilical CB is used to derive NK cells by the isolation of CD34 cells and differentiation into CD56/CD3 cells by culturing in medium contain SCF, IL-7, IL-15, and IL-2.

In some embodiments, the cell-based therapy comprises a dendritic cell-based therapy, e.g., a dendritic cell vaccine. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient. In some embodiments, immunization may be repeated multiple times if desired. Methods for harvesting, expanding, and administering dendritic cells are known in the art; see, e.g., WO2019178081. Dendritic cell vaccines (such as Sipuleucel-T, also known as APC8015 and PROVENGE®) are vaccines that involve administration of dendritic cells that act as APCs to present one or more cancer-specific antigens to the patient's immune system. In some embodiments, the vaccine comprises dendritic cells that have been exposed to one or more neoantigens. In some embodiments, the vaccine comprises dendritic cells that present one or more neoantigens, e.g., via MHC class I. In some embodiments, the dendritic cells are autologous or allogeneic to the recipient.

In some embodiments, the immunotherapy comprises a TCR-based therapy. In some embodiments, the immunotherapy comprises administration of one or more TCRs or TCR-based biologics that specifically bind a neoantigen. For example, the TCR-based therapeutic may comprise a TCR or extracellular portion thereof that specifically binds a neoantigen (e.g., as presented on a cell surface via MHC class I) as well as a moiety that binds an immune cell (e.g., a T cell), such as an antibody or antibody fragment that specifically binds a T cell surface protein or receptor (e.g., an anti-CD3 antibody or antibody fragment).

In some embodiments, the immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the immunotherapy comprises cytokine immunotherapy. Cytokine immunotherapy comprises the use of one or more cytokines that activate components of the immune system. Examples include, but are not limited to, aldesleukin (PROLEUKIN®; interleukin-2), interferon alfa-2a (ROFERON®-A), interferon alfa-2b (INTRON®-A), and peginterferon alfa-2b (PEGINTRON®).

In some embodiments, the immunotherapy comprises oncolytic virus therapy. Oncolytic virus therapy uses genetically modified viruses to replicate in and kill cancer cells, leading to the release of antigens that stimulate an immune response. In some embodiments, replication-competent oncolytic viruses expressing a tumor antigen comprise any naturally occurring (e.g. from a “field source”) or modified replication-competent oncolytic virus. In some embodiments, the oncolytic virus, in addition to expressing a tumor antigen, may be modified to increase selectivity of the virus for cancer cell. In some embodiments, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hcpadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. In some embodiments, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some embodiments, the replicative oncolytic vaccinia virus expressing a tumor antigen may be engineered to lack one or more functional genes in order to increase the cancer selectivity of the virus. In some embodiments, the oncolytic vaccinia virus is engineered to lack thymidine kinase (TK) activity. In some embodiments, the oncolytic vaccinia virus may be engineered to lack vaccinia virus growth factor (VGF). In some embodiments, the oncolytic vaccinia virus may be engineered to lack both VFG and TK activity. In some embodiments, the oncolytic vaccinia virus may be engineered to lack one or more genes involved in evading host interferon (IFN) response such as E3L, K3L, B18R, or B8R. In some embodiments, the replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen of the combination may be locally or systemically administered to a subject, e.g. via intratumoral, intraperitoneal, intravenous, intra-arterial, intramuscular, intradermal, intracranial, subcutaneous, or intranasal administration.

In some embodiments, the system may use the survival metric to assign a therapy to an individual associated with a sample. In some embodiments, the system may administer a treatment to the individual based on the survival metric. In some embodiments, the system may associate the individual with a clinical trial based on the survival metric. In some embodiments, the system may monitor the prognosis of an individual based on the survival metric. In some embodiments, the system may predict one or more clinical outcomes based on the survival metrics.

### Model Training

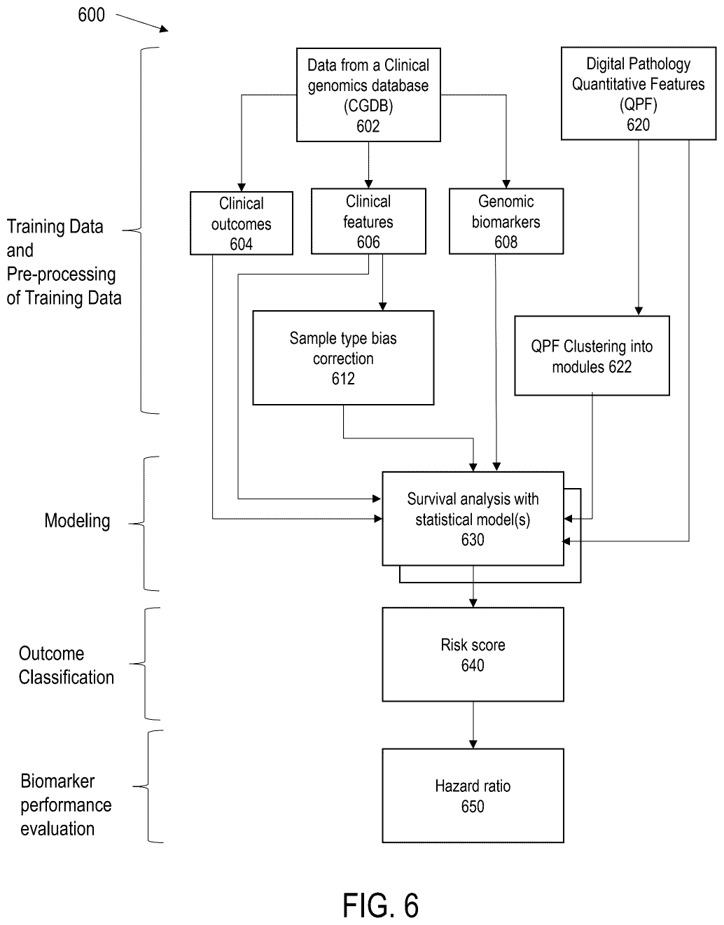

In one or more examples, the models 430, 532, and 534 may correspond to trained statistical models. FIG. 6 illustrates an exemplary process 600 for training a statistical model according to embodiments of this disclosure (e.g., models 430, 532, and 534). Process 600 can be performed, for example, using one or more electronic devices implementing a software platform. In some examples, process 600 is performed using a client-server system, and the blocks of process 600 are divided up in any manner between the server and a client device. In other examples, the blocks of process 600 are divided up between the server and multiple client devices. Thus, while portions of process 600 are described herein as being performed by particular devices of a client-server system, it will be appreciated that process 600 is not so limited. In other examples, process 600 is performed using only a client device or only multiple client devices. In process 600, some blocks are, optionally, combined, the order of some blocks is, optionally, changed, and some blocks are, optionally, omitted. In some examples, additional steps may be performed in combination with the process 600. Accordingly, the operations as illustrated (and described in greater detail below) are exemplary by nature and, as such, should not be viewed as limiting.

The system can receive training data corresponding to a plurality of training samples. This training data may be used to train the statistical model to obtain a trained statistical model. For instance, the system can receive training data 602 from a clinical genomic database (CGDB) and digital pathology quantitative pathology features (DP QPF) 620. In some instances, the clinical genomic database may comprise an inventory of genomic data and medical data corresponding to a large number of individuals (e.g., genomic data corresponding to samples obtained from hundreds or thousands of individuals). In some instances, the data 602 from the clinical genomic database includes clinical outcomes 604 (e.g., overall survival, progression free survival, time to treatment discontinuation, etc.), clinical features 606 (e.g., age, sex, ECOG status, diagnosis date, grade, stage, sample type, histologic subtype, etc.), and genomic biomarkers 608 (e.g., the presence or absence of genomic alterations, copy number alterations, rearrangements, TMB, etc.).

In some instances, the data 602 from the clinical genomics database and the digital pathology quantitative features 620 can be pre-processed to obtain additional features. For example, one or more clinical features 606 such as the sample type can be pre-processed to correct for bias in the quantitative pathology features associated with the sample type. In some instances, the way a sample is obtained can impact the predicted survival outcome for an individual, e.g., sample collection using a needle core biopsy method versus a tumor resection method. Generally, patients that are in relatively good health (e.g., robust) can have tissue samples collected via a tumor resection method. Additionally, the tumor resection method provides a larger sample and hence a larger area, which can yield a greater amount of information to determine quantitative pathology features (QPFs). Meanwhile patients that are in relatively poor health (e.g., frail) may have a tumor sample collected via a needle core biopsy, which results in a relatively smaller area of the biopsy for the extraction of QPFs. Because the health of the patient is factored into the decision of how to obtain the tissue sample and the sample-type affects determination of QPFs, it is beneficial to correct for this survival bias based on sample type.

In some examples, pre-processing the training data may include standardizing and normalizing the data features. In some examples, the system may scale continuous variables using one or more of the following techniques: standardization/zscore, min-max normalization, and log scaling. In the standardization/zscore method, continuous variables are centered by subtracting the mean from each value in the distribution and scaled by dividing each result by the standard deviation. In some examples, pre-processing the training data may include filtering some of the features to a smaller feature set. In one or more embodiments, pre-model filtering can include removing features with low prevalence, removing features with low variance, and/or selecting features that are associated with the outcome in univariate analysis.

In some examples, pre-processing the training data can include clustering the quantitative pathology features (QPFs) into modules 622. In some examples, features can be clustered via hierarchical clustering (or other clustering methods) to identify groups of features that are highly correlated. Each feature cluster may be summarized and used as an input variable in the model. This can reduce the features space and also remove correlated features that result in poor model performance. In some examples, the data from the clinical genomics database can include the characterization features 210A and 210B described above. In some examples, the digital pathology quantitative pathology features 620 can include the digital pathology features 320A, 320B, 320C described above.

Referring to FIG. 6, the training data can be input into one or more statistical models 630 to perform a survival analysis. For example, data 602 from the clinical genomics database, including clinical outcomes 604, clinical features 606, and genetic biomarkers 608, the digital pathology quantitative pathology features 620, the sample type bias correction 612, and the QPF clustering modules 622 can be input into the statistical model(s) 630. In some examples, the training data may be labeled based on the clinical outcomes 604 to train and evaluate the performance of the statistical model(s) 630 during training.

In some examples, the statistical model(s) 630 can correspond to a LASSO regression model. A LASSO regression model may associate one or more features with a patient's survival and output a risk score indicative of a likelihood of survival which can be used to determine a survival metric. For instance, by using a LASSO Cox regression model the system uses linear regression to build a relationship between predictor variables and time-to-event survival data. The risk score in this example may correspond to the linear predictor of the Cox model which is a weighted sum of the variables in the model, where the weights are the regression coefficients. In some examples, a higher risk score may be associated with a poor likelihood of survival, while a lower risk score may be associated with a better likelihood of survival. In some examples, the statistical model(s) 630 can correspond to a random forest model. For instance, the output of a survival random forest model for a particular patient may comprise a survival function which can provide survival probabilities across time. In one or more examples, example, the model may categorize patients based on associated risk groups. In one or more examples, a risk score may be calculated with a value summarizing the survival function such as probability of survival at x months (e.g., one to six months, one to twelve months, or twelve to 24 months) or area under the survival curve.

In some examples, a statistical model 630 may be trained using training data for patients who have received mono-immunotherapy. In such examples, the statistical model 630 may be applied to predict survival for both treatment with mono-immunotherapy and treatment with combination mono-immunotherapy and chemo-immunotherapy.

In some examples, separate statistical models 630 may be trained to predict a risk score for individuals treated with a mono-immunotherapy and a risk score for individuals treated with combination mono-immunotherapy and chemo-immunotherapy. In such examples, the statistical models used to predict survival for individuals treated with mono-immunotherapy may be trained using training data for patients that received mono-immunotherapy. The statistical models used to predict survival for individuals treated with chemo-immunotherapy may be trained using training data for patients that received chemo-immunotherapy.

The risk scores output by the statistical model(s) 630 may be used to determine the risk score 640. In one or more examples, e.g., when using a LASSO regression model, the risk score may be continuous. In such examples, the risk score can be categorized into risk groups using a defined threshold. In some instances, the risk scores may be compared to one or more thresholds to determine the survival metric, as discussed above (e.g., at step 110). In some instances, the survival metric and or the prediction score from the statistical models may be used in a hazard regression (e.g., Cox hazard regression) to predict a hazard ratio 650, the hazard ratio corresponding to the association between the survival of the patient and the survival metric.

In some examples, the training may be an iterative process whereby the training data is input into the statistical model and the risk score, survival metric, and or hazard ratio is compared to the actual risk score, survival metric, and or hazard ratio based on the known survival data of the patients. In this manner, weights associated with the input features, e.g., characterization features and/or QPFs, may be updated to improve the performance of the model. In one or more examples, the system may use a stepwise feature selection process to determine a threshold where covariates can be selected or removed based on whether they are statistically significant (p-value<0.05). In some examples, one or more of the input features of the model may be selected based on the determined weights. For example, if a weight associated with a particular input feature is below a predetermined threshold (indicating that the particular input features does not have a significant impact on the risk score), the particular input feature may not be selected as an input for the trained model. In one or more examples, e.g., in LASSO regression models, the system may use a penalty term to shrink the feature weights of variables that are not strongly associated with the outcome. This may lead to one or more variables being removed from the model (i.e., weights being shrunk to zero).

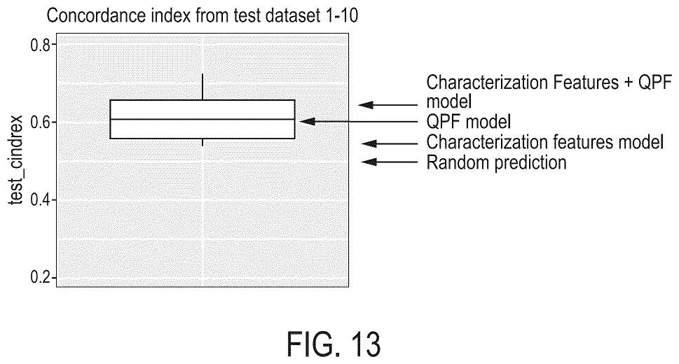

Accordingly, embodiments of the present disclosure may use machine learning models, e.g., models 430, 532, 534, and 630 to determine a risk score that can be used to determine a survival metric indicative of a likelihood of survival of a patient and/or a response of a patient when treated with mono-immunotherapy and/or chemo-immunotherapy. Due to the computational complexity involved in pre-processing and analyzing the genomic information and digital pathology information, statistical models in accordance with embodiment of the present disclosure are a valuable tool to determine the risk score and survival metric. FIG. 13 illustrates an exemplary box plot showing a concordance index corresponding to models that were trained and implemented in accordance with embodiments of this disclosure. As shown in the figure, a random prediction would have a concordance of about 0.5 or 50% (e.g., we would expect a random prediction to be accurate about 50% of the time). A statistical model implemented in accordance with embodiments of the present disclosure that is trained using characterization features and provided with characterization features as inputs improves upon the accuracy of a random prediction. A statistical model implemented in accordance with embodiments of the present disclosure that is trained using QPFs and is provided with QPFs improves upon the accuracy of the random prediction and the genomic model. Combining the predictive power of the genomic features and the quantitative features, embodiments of this disclosure provide a more accurate way to predict the survival of an individual compared to survival predictions based on these features alone.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) performing a methylation conversion reaction to convert, e.g., non-methylated cytosine to uracil, (v) amplifying the nucleic acid molecules (e.g., using a polymerase chain reaction (PCR) amplification technique, a non-PCR amplification technique, or an isothermal amplification technique), (vi) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vii) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, (viii) combining the nucleic acid sequence data (including, e.g., variant data, copy number data, methylation status data, etc., of the sequenced nucleic acid molecules) with other biomarker data modalities including, but not limited to, proteomics-based biomarker data (e.g., the detection of specific polypeptides, such as proteins) or fragmentomics-based biomarker data (e.g., the detection of certain attributes related to nucleic acid fragments, such as fragment size or the sequences of fragment ends), to determine, for example, the presence of ctDNA in the sample and/or to determine a diagnostic, prognostic, and/or treatment response prediction for the subject, and (ix) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA). In some instances, the cell-free DNA (cfDNA), or a portion thereof, may comprise circulating tumor DNA (ctDNA). In some instances, the liquid biopsy sample may comprise a combination of cell-free DNA (cfDNA) and circulating tumor DNA (ctDNA).

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for determining a survival metric may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for determining a survival metric may be used to predict genetic disorders in fetal DNA. (e.g., for invasive or non-invasive prenatal testing). For example, sequence read data obtained by sequencing fetal DNA extracted from samples obtained using invasive amniocentesis, chorionic villus sampling (cVS), or fetal umbilical cord sampling techniques, or obtained using non-invasive sampling of cell-free DNA (cfDNA) samples (which comprises a mix of maternal cfDNA and fetal cfDNA), may be processed according to the disclosed methods to identify variants, e.g., copy number alterations, associated with, e.g., Down Syndrome (trisomy 21), trisomy 18, trisomy 13, and extra or missing copies of the X and Y chromosomes.

In some instances, the disclosed methods for determining a survival metric may be used to select a subject (e.g., a patient) for a clinical trial based on the survival metric value determined for one or more gene loci. In some instances, patient selection for clinical trials based on, e.g., determination of the survival metric, may accelerate the development of targeted therapies and improve the healthcare outcomes for treatment decisions.

In some instances, the disclosed methods for determining a survival metric may be used to select an appropriate therapy or treatment (e.g., an anti-cancer therapy or anti-cancer treatment) for a subject. In some instances, for example, the anti-cancer therapy or treatment may comprise use of a poly(ADP-ribose) polymerase inhibitor (PARPi), a platinum compound, chemotherapy, radiation therapy, a targeted therapy, an immunotherapy, a neoantigen-based therapy, surgery, or any combination thereof.

In some instances, the anti-cancer therapy or treatment may comprise a targeted anti-cancer therapy or treatment (e.g., a monoclonal antibody-based therapy, an enzyme inhibitor-based therapy, an antibody-drug conjugate therapy, a hormone therapy, and/or a targeted radiotherapy) that targets specific molecules required for cancer cell growth, division, and spreading. In some instances, the targeted anti-cancer therapy or treatment may comprise abemaciclib (Verzenio), abiraterone acetate (Zytiga), acalabrutinib (Calquence), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the anti-cancer therapy or treatment may comprise an immunotherapy (e.g., a cancer treatment that acts by stimulating the immune system to fight cancer). In some instances, the immunotherapy can be, for example, an immune system modulator (e.g., a cytokine, such as an interferon or interleukin), an immune checkpoint inhibitor (such as an anti-PD-1 or anti-PD-L1 antibody), a T-cell transfer therapy (e.g., a tumor infiltrating lymphocyte (TIL) therapy in lymphocytes extracted from a patient's tumor are selected for their ability to recognize tumor cells and propagated prior to reintroduction into the patient, or a CAR T-cell therapy in which a patient's T-cells are modified to express the CAR protein prior to reintroduction into the patient), a monoclonal antibody-based therapy (e.g., a monoclonal antibody that binds to cell surface markers on cancer cells to facilitate recognition by the immune system), or a cancer treatment vaccine (e.g., a vaccine based on tumor cells, tumor-associated neoantigens, or dendritic cells, etc., that stimulates the immune system to fight cancer).

In some instances, the anti-cancer therapy or treatment may comprise a neoantigen-based therapy. Non-limiting examples of neoantigen-based therapies include T-cell receptor (TCR) engineered T-cell (TCR-T) therapies, chimeric antigen receptor T-cell (CAR-T) therapies, TCR bispecific antibody therapies, and cancer vaccines. TCR-T therapies are produced by genetically engineering a patient's T-cells to express T-cell receptors that are specific to neoantigens of interest, and then infusing them back into the patient. CAR-T therapies are produced by genetically engineering a patient's T-cells to express chimeric antigen receptor molecules which contain an intracellular signaling and co-signaling domain as well as an extracellular antigen-binding domain; CAR-T therapies don't always rely on neoantigen presentation, but can be designed to be directed towards neoantigens. TCR bispecific antibody therapies are small, engineered antibody molecules that comprise a neoantigen-specific TCR on one end and a CD3-directed single-chain variable fragment on the other end. Cancer vaccines can include RNA molecules, DNA molecules, peptides, or a combination thereof that are designed to boost the immune system's ability to find and destroy neoantigen-presenting cells.

In some instances, the disclosed methods for determining a survival metric may be used in treating a disease (e.g., a cancer) in a subject. For example, in response to determining the survival metric using any of the methods disclosed herein, an effective amount of an anti-cancer therapy or anti-cancer treatment may be administered to the subject.

In some instances, the disclosed methods for determining a survival metric may be used for monitoring disease progression or recurrence (e.g., cancer or tumor progression or recurrence) in a subject. For example, in some instances, the methods may be used to determine a survival metric in a first sample obtained from the subject at a first time point, and used to determine a survival metric in a second sample obtained from the subject at a second time point, where comparison of the first determination of the survival metric and the second determination of the survival metric allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the determination of a survival metric.

In some instances, the value of the survival metric determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for determining a survival metric may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for determining a survival metric as part of a genomic profiling process (or inclusion of the output from the disclosed methods for determining a survival metric as part of the genomic profile of the subject) can improve the validity of, e.g., disease detection calls and treatment decisions, made on the basis of the genomic profile by, for example, independently determining a survival metric for in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo (dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMOL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1): 35-42; Masuda, et al., (1999) Nucleic Acids Res. 27(22): 4436-4443; Specht, et al., (2001) Am J Pathol. 158(2): 419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11): 903-5; Hodges, E. et al. (2007) Nat. Genet. 39(12): 1522-7; and Okou, D. T. et al. (2007) Nat. Methods 4(11): 907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLID, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1): 195-197), the Striped Smith-Waterman algorithm (see, e.g., Farrar (2007), “Striped Smith-Waterman Speeds Database Searches Six Times Over Other SIMD Implementations”, Bioinformatics 23(2): 156-161), the Needleman-Wunsch algorithm (Needleman, et al. (1970) “A General Method Applicable to the Search for Similarities in the Amino Acid Sequence of Two Proteins”, J. Molecular Biology 48(3): 443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized.

In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. CàT in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Alignment of Methyl-Seq Sequence Reads

In some instances, the methods may include the use of an alignment method optimized for aligning sequence reads for DNA that has been converted using, e.g., a bisulfite reaction, to convert unmethylated cytosine residues to uracil (which is interpreted as a thymine in sequencing results). In some instances, sequence reads may be aligned to two genomes in silico, e.g., converted and unconverted versions of the reference genome, using such alignment tools. Methylation occurs primarily at CpG sites, but may also occur less frequently at non-CpG sites (e.g., CHG or CHH sites).

In some instances, the sequence read data may be obtained using a nucleic acid sequencing method comprising the use of a bisulfite- or enzymatic-conversion reaction (e.g., during library preparation) to convert non-methylated cytosine to uracil (see, e.g., Li, et al. (2011), “DNA Methylation Detection: Bisulfite Genomic Sequencing Analysis”, Methods Mol. Biol. 791:11-21).

In some instances, the sequence read data may be obtained using a nucleic acid sequencing method comprising the use of alternative chemical and/or enzymatic reactions (e.g., during library preparation) to convert non-methylated cytosine to uracil (or to convert methylated cytosine to dihydrouracil). For example, enzymatic deamination of non-methylated cytosine using APOBEC to form uracil can be performed using, e.g., the Enzymatic Methyl-seq Kit from New England BioLabs (Ipswich, MA) which uses prior treatment with ten-eleven translocation methylcytosine dioxygenase 2 (TET2) to oxidize 5-mC and 5-hmC, thereby providing greater protection of the methylated cytosine from deamination by APOBEC). Liu, et al. (2019) recently described a bisulfite-free and base-level-resolution sequencing-based method, TET-Assisted Pyridine borane Sequencing (TAPS), for detection of 5mC and 5hmC. The method combines ten-eleven translocation methylcytosine dioxygenase (TET)-mediated oxidation of 5mC and 5hmC to 5-carboxylcytosine (5caC) with pyridine borane reduction of 5caC to dihydrouracil (DHU). Subsequent PCR amplification converts DHU to thymine, thereby enabling conversion of methylated cytosines to thymine (Liu, et al. (2019), “Bisulfite-Free Direct Detection of 5-Methylcytosine and 5-Hydroxymethylcytosine at Base Resolution”, Nature Biotechnology, vol. 37, pp. 424-429).

In some instances, the sequence read data may be obtained using a nucleic acid sequencing method comprising the use of Methylated DNA Immunoprecipitation (MeDIP).

Examples of alignment tools optimized for aligning sequence reads for converted DNA include, but are not limited to, NovoAlign (Novocraft Technologies, Selangor, Malaysia), and the Bismark tool (Krueger, et al. (2011), “Bismark: A Flexible Aligner and Methylation Caller for Bisulfite-Seq Applications”, Bioinformatics 27(11): 1571-1572).

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6): 847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9): 1297-303; Ye, K., et al., Bioinformatics, 2009; 25(21): 2865-71; Lunter, G., and Goodson, M., Genome Res. 2011; 21(6): 936-9; and Li, H., et al. (2009), Bioinformatics 25(16): 2078-9.

Methods for generating indel calls and individual-level genotype likelihoods include, e.g., the Dindel algorithm (Albers, C. A., et al., Genome Res. 2011; 21(6): 961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6): 952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 Mar. 15; 26(6): 730-736) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of exemplary nucleic acid sequencing methods, mutation calling methods, and methods for analysis of genetic variants is provided in, e.g., U.S. Pat. Nos. 9,340,830, 9,792,403, 11,136,619, 11,118,213, and International Patent Application Publication No. WO 2020/236941, the entire contents of each of which is incorporated herein by reference.

### Methylation Status Calling

In some instances, the methods described herein may comprise the use of a methylation status calling method, e.g., to call the methylation status of the CpG sites based on the sequence reads and fragments (complementary pairs of forward and reverse sequence reads) derived from DNA that has been subjected to a chemical or enzymatic conversion reaction, e.g., to convert unmethylated cytosine residues to uracil (which is interpreted as a thymine in sequencing results). Examples of such methylation status calling tools include, but are not limited to, the Bismark tool (Krueger, et al. (2011), “Bismark: A Flexible Aligner and Methylation Caller for Bisulfite-Seq Applications”, Bioinformatics 27(11): 1571-1572), TARGOMICS (Garinet, et al. (2017), “Calling Chromosome Alterations, DNA Methylation Statuses, and Mutations in Tumors by Simple Targeted Next-Generation Sequencing—A Solution for Transferring Integrated Pangenomic Studies into Routine Practice?”, J. Molecular Diagnostics 19(5): 776-787), Bicycle (Grana, et al. (2018) “Bicycle: A Bioinformatics Pipeline to Analyze Bisulfite Sequencing Data”, Bioinformatics 34(8): 1414-5), SMAP (Gao, et al. (2015), “SMAP: A Streamlined Methylation Analysis Pipeline for Bisulfite Sequencing”, Gigascience 4:29), and MeDUSA (Wilson, et al. (2016), “Computational Analysis and Integration of MeDIP-Seq Methylome Data”, in: Kulski J K, editor, Next Generation Sequencing: Advances, Applications and Challenges. Rijeka: InTech, p. 153-69). See also, Rauluseviciute, et al. (2019), “DNA Methylation Data by Sequencing: Experimental Approaches and Recommendations for Tools and Pipelines for Data Analysis”, Clinical Epigenetics 11:193.

### Systems

Also disclosed herein are systems designed to implement any of the disclosed methods for determining a survival metric of a sample from a subject. The systems may comprise, e.g., one or more processors, and a memory unit communicatively coupled to the one or more processors and configured to store instructions that, when executed by the one or more processors, cause the system to: receive sequence read data for the plurality of sequence reads associated with the sample from the subject; selecting a plurality of sequence reads from the sequence read data; determine one or more characterization features based on the plurality of sequence reads; receive one or more digital pathology features; input the one or more characterization features and the one or more digital pathology features into one or more statistical models; and predict a survival metric indicative of a likelihood of survival of the individual, based on outputs of the one or more statistical models.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLID) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for determining a survival metric of any of a variety of samples as described herein (e.g., a tissue sample, biopsy sample, hematological sample, or liquid biopsy sample derived from the subject).

In some instances, the plurality of gene loci for which sequencing data is processed to determine the survival metric may comprise at least 1, 2, 3, 4, 5, 6, 7, 8, 9, 10, or more than 10 gene loci.

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of a survival metric is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Machine Learning

Any of a variety of machine learning approaches & algorithms (where a machine learning model, as referred to herein, comprises a trained machine learning algorithm) may be used in implementing the disclosed methods. For example, the machine learning model may comprise a supervised learning model (i.e., a model trained using labeled sets of training data), an unsupervised learning model (i.e., a model trained using unlabeled sets of training data), a semi-supervised learning model (i.e., a model trained using a combination of labeled and unlabeled training data), a self-supervised learning model, or any combination thereof. In some examples, the machine learning model can comprise a deep learning model (i.e., a model comprising many layers of coupled “nodes” that may be trained in a supervised, unsupervised, or semi-supervised manner).

In some instances, one or more machine learning models (e.g., 1, 2, 3, 4, 5, 6, 7, 8, 9, 10, or more than 10 machine learning models), or a combination thereof, may be utilized to implement the disclosed methods.

In some instances, the one or more machine learning models may comprise statistical methods for analyzing data. The machine learning models may be used for classification and/or regression of data. The machine learning models can include, for example, neural networks, support vector machines, decision trees, ensemble learning (e.g., bagging-based learning, such as random forest, and/or boosting-based learning), k-nearest neighbors algorithms, linear regression-based models, and/or logistic regression-based models. The machine learning models can comprise regularization, such as L1 regularization and/or L2 regularization. The machine learning models can include the use of dimensionality reduction techniques (e.g., principal component analysis, matrix factorization techniques, and/or autoencoders) and/or clustering techniques (e.g., hierarchical clustering, k-means clustering, distribution-based clustering, such as Gaussian mixture models, or density-based clustering, such as DBSCAN or OPTICS). The one or more machine learning models can comprise solving, e.g., optimizing, an objective function over multiple iterations based on a training data set. The iterative solving approach can be used even when the machine learning model comprises a model for which there exists a closed-form solution (e.g., linear regression).

In some instances, the machine learning models can comprise artificial neural networks (ANNs), e.g., deep learning models. For example, the one or more machine learning models/algorithms used for implementing the disclosed methods may include an ANN which can comprise any of a variety of computational motifs/architectures known to those of skill in the art, including, but not limited to, feedforward connections (e.g., skip connections), recurrent connections, fully connected layers, convolutional layers, and/or pooling functions (e.g., attention, including self-attention). The artificial neural networks can comprise differentiable non-linear functions trained by backpropagation.

Artificial neural networks, e.g., deep learning models, generally comprise an interconnected group of nodes organized into multiple layers of nodes. For example, the ANN architecture may comprise at least an input layer, one or more hidden layers (i.e., intermediate layers), and an output layer. The ANN or deep learning model may comprise any total number of layers (e.g., 3, 4, 5, 6, 7, 8, 9, 10, 15, 20, or more than 20 layers in total), and any number of hidden layers (e.g., 1, 2, 3, 4, 5, 6, 7, 8, 9, 10, 15, 20, or more than 20 hidden layers), where the hidden layers function as trainable feature extractors that allow mapping of a set of input data to a preferred output value or set of output values. Each layer of the neural network comprises a plurality of nodes (e.g., at least 10, 25, 50, 75 100, 200, 300, 400, 500, 600, 700, 800, 900, 1000, 2000, 3000, 4000, 5000, 6000, 7000, 8000, 9000, 10,000, or more than 10,000 nodes). A node receives input data (e.g., genomic feature data (such as variant sequence data, methylation status data, etc.), non-genomic feature data (e.g., digital pathology image feature data), or other types of input data (e.g., patient-specific clinical data)) that comes either directly from one or more input data nodes or from the output of one or more nodes in previous layers, and performs a specific operation, e.g., a summation operation. In some cases, a connection from an input to a node is associated with a weight (or weighting factor). In some cases, the node may, for example, sum up the products of all pairs of inputs, Xᵢ, and their associated weights, Wᵢ. In some cases, the weighted sum is offset with a bias, b. In some cases, the output of a node may be gated using a threshold or activation function, f, where f may be a linear or non-linear function. The activation function may be, for example, a rectified linear unit (ReLU) activation function or other function such as a saturating hyperbolic tangent, identity, binary step, logistic, arcTan, softsign, parameteric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network (or other machine learning architecture), can be “taught” or “learned” in a training phase using one or more sets of training data (e.g., 1, 2, 3, 4, 5, or more than 5 sets of training data) and a specified training approach configured to solve, e.g., minimize, a loss function. For example, the adjustable parameters for an ANN (e.g., deep learning model) may be determined based on input data from a training data set using an iterative solver (such as a gradient-based method, e.g., backpropagation), so that the output value(s) that the ANN computes (e.g., a classification of a sample or a prediction of a disease outcome) are consistent with the examples included in the training data set. The training of the model (i.e., determination of the adjustable parameters of the model using an iterative solver) may or may not be performed using the same hardware as that used for deployment of the trained model.

In some instances, the disclosed methods may comprise retraining any of the machine learning models (e.g., iteratively retraining a previously trained model using one or more training data sets that differ from those used to train the model initially). In some instances, retraining the machine learning model may comprise using a continuous, e.g., online, machine learning model, i.e., where the model is periodically or continuously updated or retrained based on new training data. The new training data may be provided by, e.g., a single deployed local operational system, a plurality of deployed local operational systems, or a plurality of deployed, geographically-distributed operational systems. In some instances, the disclosed methods may employ, for example, pre-trained ANNs, and the pre-trained ANNs can be fine-tuned according to an additional dataset that is inputted into the pre-trained ANN.

### Computer Systems and Networks

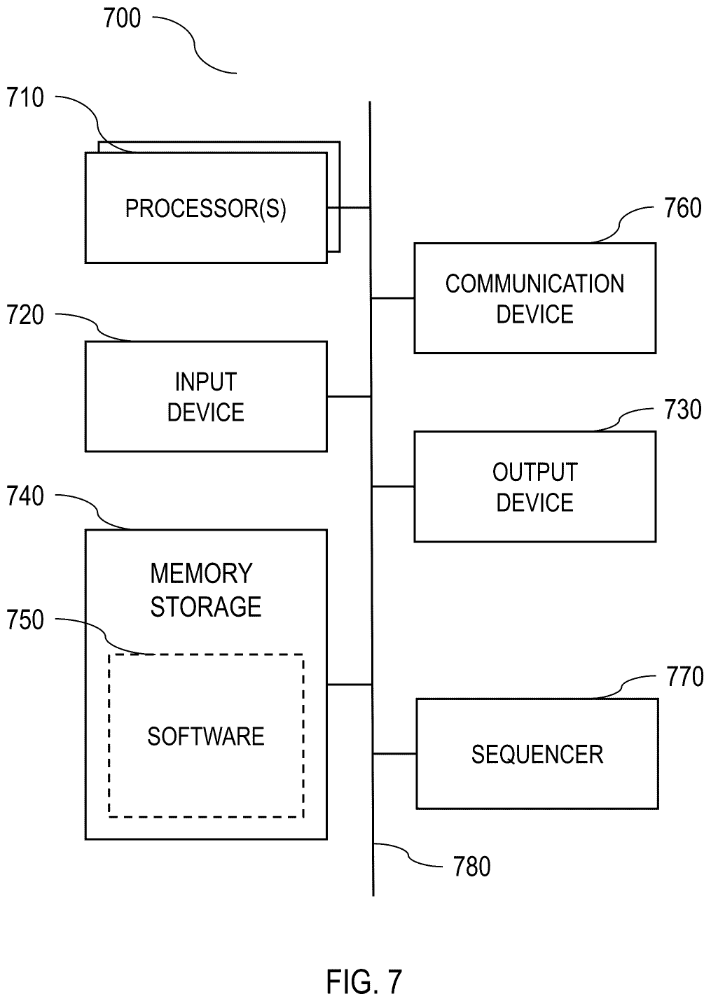

FIG. 7 illustrates an example of a computing device or system in accordance with one embodiment. Device 700 can be a host computer connected to a network. Device 700 can be a client computer or a server. As shown in FIG. 7, device 700 can be any suitable type of microprocessor-based device, such as a personal computer, workstation, server or handheld computing device (portable electronic device) such as a phone or tablet. The device can include, for example, one or more processor(s) 710, input devices 720, output devices 730, memory or storage devices 740, communication devices 760, and nucleic acid sequencers 770. Software 750 residing in memory or storage device 740 may comprise, e.g., an operating system as well as software for executing the methods described herein. Input device 720 and output device 730 can generally correspond to those described herein, and can either be connectable or integrated with the computer.

Input device 720 can be any suitable device that provides input, such as a touch screen, keyboard or keypad, mouse, or voice-recognition device. Output device 730 can be any suitable device that provides output, such as a touch screen, haptics device, or speaker.

Storage 740 can be any suitable device that provides storage (e.g., an electrical, magnetic or optical memory including a RAM (volatile and non-volatile), cache, hard drive, or removable storage disk). Communication device 760 can include any suitable device capable of transmitting and receiving signals over a network, such as a network interface chip or device. The components of the computer can be connected in any suitable manner, such as via a wired media (e.g., a physical system bus 780, Ethernet connection, or any other wire transfer technology) or wirelessly (e.g., Bluetooth®, Wi-Fi®, or any other wireless technology).

Software module 750, which can be stored as executable instructions in storage 740 and executed by processor(s) 710, can include, for example, an operating system and/or the processes that embody the functionality of the methods of the present disclosure (e.g., as embodied in the devices as described herein).

Software module 750 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 740, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 750 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

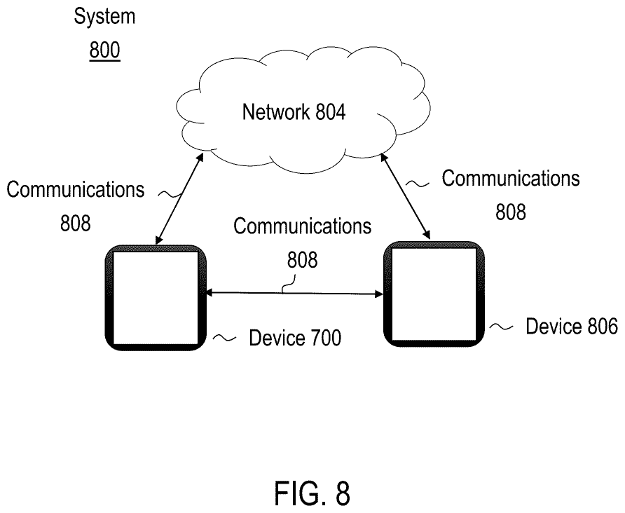

Device 700 may be connected to a network (e.g., network 804, as shown in FIG. 8 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 700 can be implemented using any operating system, e.g., an operating system suitable for operating on the network. Software module 750 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 710.

Device 700 can further include a sequencer 770, which can be any suitable nucleic acid sequencing instrument.

FIG. 8 illustrates an example of a computing system in accordance with one embodiment. In system 800, device 700 (e.g., as described above and illustrated in FIG. 7) is connected to network 804, which is also connected to device 806. In some embodiments, device 806 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio® RS system.

Devices 700 and 806 may communicate, e.g., using suitable communication interfaces via network 804, such as a Local Area Network (LAN), Virtual Private Network (VPN), or the Internet. In some embodiments, network 804 can be, for example, the Internet, an intranet, a virtual private network, a cloud network, a wired network, or a wireless network. Devices 700 and 806 may communicate, in part or in whole, via wireless or hardwired communications, such as Ethernet, IEEE 802.11b wireless, or the like. Additionally, devices 700 and 806 may communicate, e.g., using suitable communication interfaces, via a second network, such as a mobile/cellular network. Communication between devices 700 and 806 may further include or communicate with various servers such as a mail server, mobile server, media server, telephone server, and the like. In some embodiments, Devices 700 and 806 can communicate directly (instead of, or in addition to, communicating via network 804), e.g., via wireless or hardwired communications, such as Ethernet, IEEE 802.11b wireless, or the like. In some embodiments, devices 700 and 806 communicate via communications 808, which can be a direct connection or can occur via a network (e.g., network 804).

One or all of devices 700 and 806 generally include logic (e.g., http web server logic) or are programmed to format data, accessed from local or remote databases or other sources of data and content, for providing and/or receiving information via network 804 according to various examples described herein.

### EXAMPLES

This section provides non-limiting examples of determining a survival metric in accordance with one or more embodiments of the present disclosure. In some examples, the survival metric may be based on characterization features (e.g., characterization features as discussed above with respect to FIGS. 2A-2B). In some examples, the survival metric may be based on digital pathology quantitative pathology features (QPFs) (e.g., QPFs as discussed above with respect to FIGS. 3A-3C). In some examples, the survival metric may be based on characterization features and QPFs.

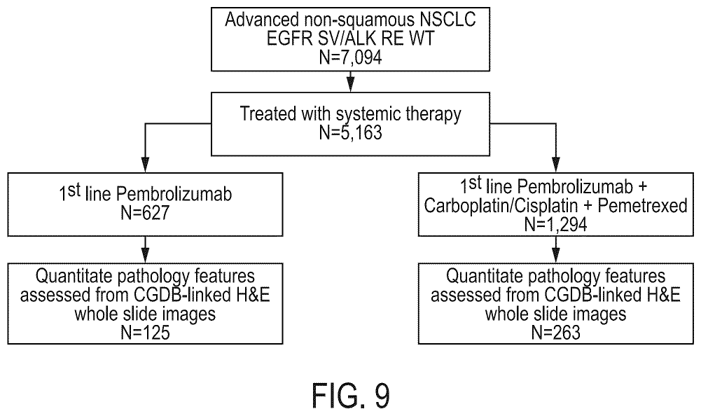

FIG. 9 illustrates exemplary data used to train one or more statistical models used to determine a survival metric in accordance with one or more embodiments of the present disclosure. As shown in the figure, the exemplary data corresponds to data from non-squamous non-small cell lung cancer patients without actionable alterations in EGFR and ALK (n=7,094) that were treated with a systemic therapy (n=5,163). From this data 627 patients were treated with Pembrolizumab as a first line mono-immunotherapy. Of these 627 patients, 125 of these samples were assessed (e.g., stained) to determine digital pathology QPFs. 1,294 patients were treated with were treated with a combination therapy with Pembrolizumab as a first line mono-immunotherapy along with a chemo-immunotherapy such as Carboplatin/Cisplatin and/or Pemetrexed. Of these 1,294 patients, 263 of these samples were assessed (e.g., stained) to determine digital pathology QPFs.

### Prediction Model Based on Characterization Features

According to a first example, the data from the 627 patients treated with Pembrolizumab were used to determine a statistical model to predict a risk score based on characterization features and the known survival outcomes of the patients. In this example, a statistical model (e.g., LASSO Cox regression) was used to build a prediction model for an overall survival of a patient after treatment with a first line treatment with a mono-immunotherapy (e.g., Pembrolizumab). Characterization features including whether a genomic alteration (e.g., short variant alteration, copy number alteration, rearrangement alteration) was present or absent and a TMB value was used to determine the survival metric. The statistical model was trained using an 80:20 training-validation split and supervised learning to determine one or more weights associated with the characterization features. Other training-validation splits may be used without departing from the scope of this disclosure. In this example, the characterization features included in the model may correspond to the characterization features 210B.

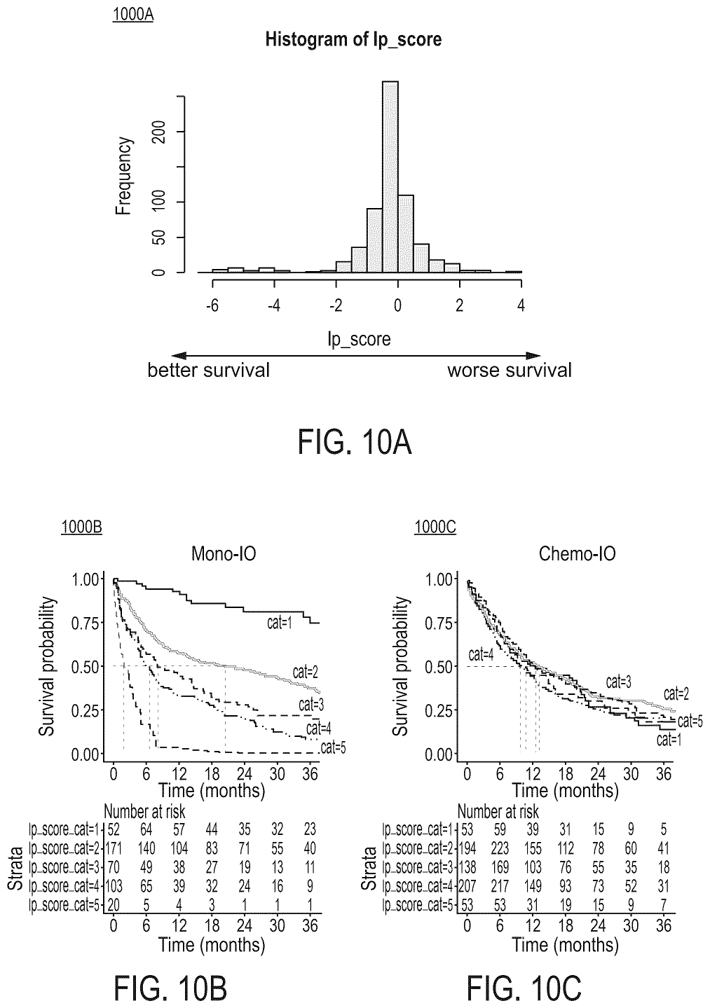

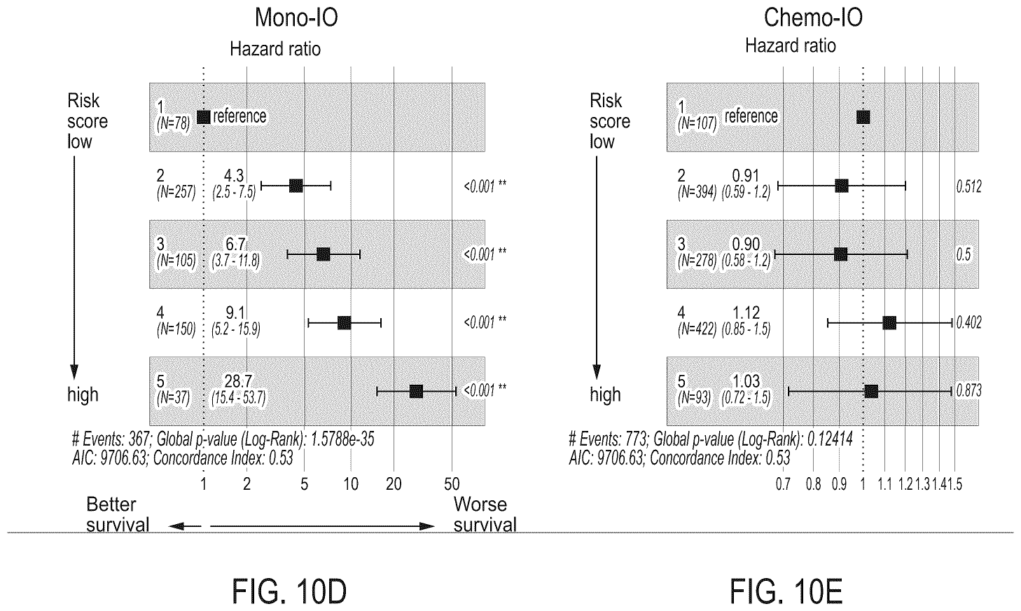

FIG. 10A illustrates a histogram 1000A of the determined survival score for the 672 samples. As shown in the figure, a higher survival score was associated with a worse survival, while a lower survival score was associated with a better survival. FIG. 10B illustrates a survival plot 1000B for patients (e.g., n=627) that were treated with a mono-immunotherapy. FIG. 10C illustrates a survival plot 1000C for patients (e.g., n=1,294) that were treated with a mono-immunotherapy in combination with a chemo-immunotherapy. In this example, the same statistical model was applied to the data for patients treated with a mono-immunotherapy and patients treated with a mono-immunotherapy in combination with a chemo-immunotherapy. As shown in the plots, the patients were stratified to determine a survival metric using one or more thresholds based on the risk score determined by the model. For instance, the first group corresponds to patients with a risk score below negative one, the second group corresponds to patients with a risk score between negative one and zero, the third group corresponds to patients with a risk score of zero, the fourth group corresponds to patients with a risk score between zero and one, and the fifth group corresponds to patients with a risk score above one. While the patients were stratified into five groups, a skilled artisan would understand that the patients may be stratified into less groups (two to four) or more groups (e.g., six or more) without departing from the scope of this disclosure.

A comparison of plots 1000B and 1000C yield insights into the impact of treatment on survival of a patient. For instance, patients in the first group (e.g., corresponding to a low risk score) had a better overall survival when treated with a mono-immunotherapy as opposed to a combination mono-immunotherapy and chemo-immunotherapy treatment. On the other hand, patients in the fourth and fifth groups (e.g., with higher risk scores) had a better overall survival when treated with chemo-immunotherapy as opposed to mono-immunotherapy alone. Based on these results, embodiments of the present disclosure may be used to predict the survival outcomes and/or a survival metric of new patient. For instance, the system may receive data associated with a new patient and determine one or more characterization features. These characterization features can be input into the statistical model to determine the risk score. Based on the risk score, the system may determine a survival metric and may further determine whether the patient is a more suitable candidate for mono-immunotherapy and/or chemotherapy.

FIG. 10D illustrates the hazard ratio for the patients treated with mono-immunotherapy stratified based on the risk score. FIG. 10E illustrates the hazard ratio for the patients treated with chemo-immunotherapy stratified based on the risk score. In some examples, the hazard ratio indicates the fold increase in hazard (risk) of death relative for a particular risk group compared to the reference risk group as shown in FIG. 10D, patients treated with mono-immunotherapy with a lower risk score had a better survival than patients in the second through fifth groups (e.g., with a higher risk score). As shown in FIG. 10E, the risk score alone and/or survival metric was not predictive of the survival outcomes for patients treated with chemo-immunotherapy.

### Prediction Model Based on Quantitative Pathology Features

According to a second example, the data from patients with tissue resection samples (n=52) that were treated with Pembrolizumab were used to determine a model to predict a risk score for patients based on QPF data and the known survival outcomes of the patients. Due to the larger area and greater number of identifiable QPFs associated with tissue resection samples (e.g., as opposed to needle core biopsy samples) the tissue resection samples were used to build the statistical model. In one or more examples the larger tissue area associated with tissue resections may also reduce measurement error compared to needle core biopsy samples.

In this example, a statistical model (e.g., LASSO Cox regression) was used to build the statistical model to determine a risk score for an overall survival of a patient after treatment with a first line treatment with a mono-immunotherapy (e.g., Pembrolizumab). Quantitative pathology features (QPFs) as discussed above with respect to FIGS. 3A-3C were used to determine a statistical model to predict the risk score. The statistical model was trained using an 80:20 training-validation split and supervised learning to determine one or more weights associated with the QPFs. Although other training-validation splits may be used without departing from the scope of this disclosure. In this example, the characterization features included in the model correspond to the QPF features 310C.

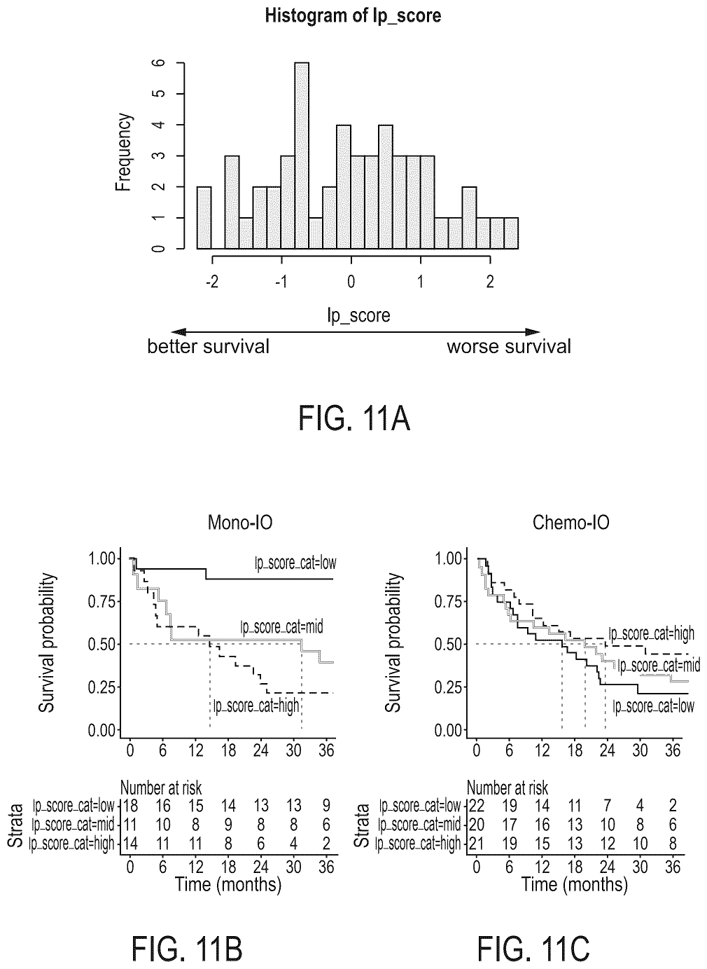

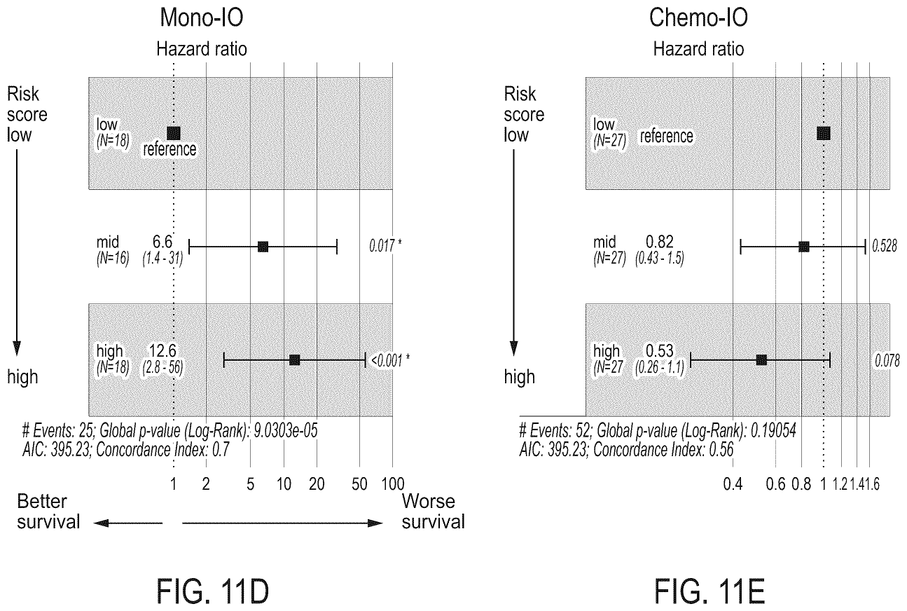

FIG. 11A illustrates a histogram 1100A of the determined survival score for the 125 samples. As shown in the figure, a higher survival score was associated with a worse survival, while a lower survival score was associated with a better survival. FIG. 11B illustrates a survival plot 1100B for patients (e.g., n=125) that were treated with a mono-immunotherapy. FIG. 11C illustrates a survival plot 1100C for patients (e.g., n=263) that were treated with a mono-immunotherapy in combination with a chemo-immunotherapy. In this example, the same statistical model was applied to the data for patients treated with a mono-immunotherapy and patients treated with a mono-immunotherapy in combination with a chemo-immunotherapy. As shown in the plots, the patients were stratified using one or more thresholds based on the risk score determined by the model to determine a survival metric. In one or more examples, tertiles may be used to determine the thresholds. While the patients were stratified into three groups, a skilled artisan would understand that the patients may be stratified into more or less groups without departing from the scope of this disclosure.

A comparison of plots 1100B and 1100C yield insights into the impact of treatment on survival of a patient. For instance, patients in the first group (e.g., corresponding to a low risk score) had a better overall survival when treated with a mono-immunotherapy as opposed to treatment with combination mono-immunotherapy and chemo-immunotherapy. Based on these results, embodiments of the present disclosure may be used to predict the survival outcomes and/or a survival metric of new patient. For instance, the system may receive digital pathology data associated with a new patient and determine one or more QPFs. These QPFs can be input into the statistical model to determine the risk score. Based on the risk score, the system may determine a survival metric and may further determine whether the patient is a more suitable candidate for mono-immunotherapy and/or chemotherapy. For instance, if the patient has a low risk score, the system may determine that the patient is a suitable candidate for mono-immunotherapy.

FIG. 11D illustrates the hazard ratio for the patients treated with mono-immunotherapy stratified based on the risk score. FIG. 11E illustrates the hazard ratio for the patients treated with chemo-immunotherapy stratified based on the risk score. As shown in FIG. 11D, patients treated with mono-immunotherapy in the second and third groups (e.g., associated with a higher risk score) are associated with a worse survival. As shown in FIG. 11E, the risk score alone was not predictive of the survival outcomes for patients treated with chemo-immunotherapy.

### Prediction Model Based on Characterization Features and Quantitative Pathology Features

According to a third example, the data from patients with tissue resection samples (n=52) that were treated with Pembrolizumab were used to determine a model to predict a risk score for patients based on characterization features, QPF data, and the known survival outcomes of the patients. In this example, a statistical model (e.g., LASSO Cox regression) was used to build a prediction model for an overall survival of a patient after treatment with a first line treatment with a mono-immunotherapy (e.g., Pembrolizumab). Characterization features (e.g., as discussed above with respect to FIGS. 2A and 2B) and quantitative pathology features (QPFs) (e.g., as discussed above with respect to FIGS. 3A-3C) were used to determine a statistical model to predict the risk score. The statistical model was trained using an 80:20 training-validation split and supervised learning to determine one or more weights associated with the characterization features and the QPFs. Table II provides exemplary features and corresponding weights determined using methods in accordance with embodiments of this disclosure. It will be appreciated that other training-validation splits may be used without departing from the scope of this disclosure.

TABLE II

| Feature | Coefficient |
|---|---|
| COUNT PROP [[GRANULOCYTE CELLS] OVER [IMMUNE | 0.15069472 |
| CELLS]] IN [TUMOR]_HE | |
| CELL CLUSTERING: CLUSTER DISPERSION SD | −0.3699977 |
| LYMPHOCYTE IN CANCER EPITHELIUM | |
| (UNVALIDATED)_HE | |
| CELL CLUSTERING: CLUSTER SIZE STANDARD | −0.3229181 |
| DEVIATION CANCER CELL IN CANCER EPITHELIUM | |
| (UNVALIDATED)_HE | |
| REGION PROPERTIES: SOLIDITY OF LARGEST REGION | −0.1789524 |
| OF CANCER STROMA_HE | |
| TOTAL EULER NUMBER OF CANCER EPITHELIUM_HE | −0.1784577 |
| BCOR | 1.28174555 |
| BRAF | 0.18130075 |
| FAM123B | 0.68131902 |
| GATA3 | −0.1523311 |
| HGF | 0.53776083 |
| IRF2 | −0.6572157 |
| NF2 | 2.70956522 |
| NPM1 | −0.2711917 |
| PIK3CA | 1.34885379 |
| RAD54L | 1.87931309 |
| SDHC | −0.0195766 |
| SMAD4 | −0.7574351 |
| tmb10 | −0.0225132 |

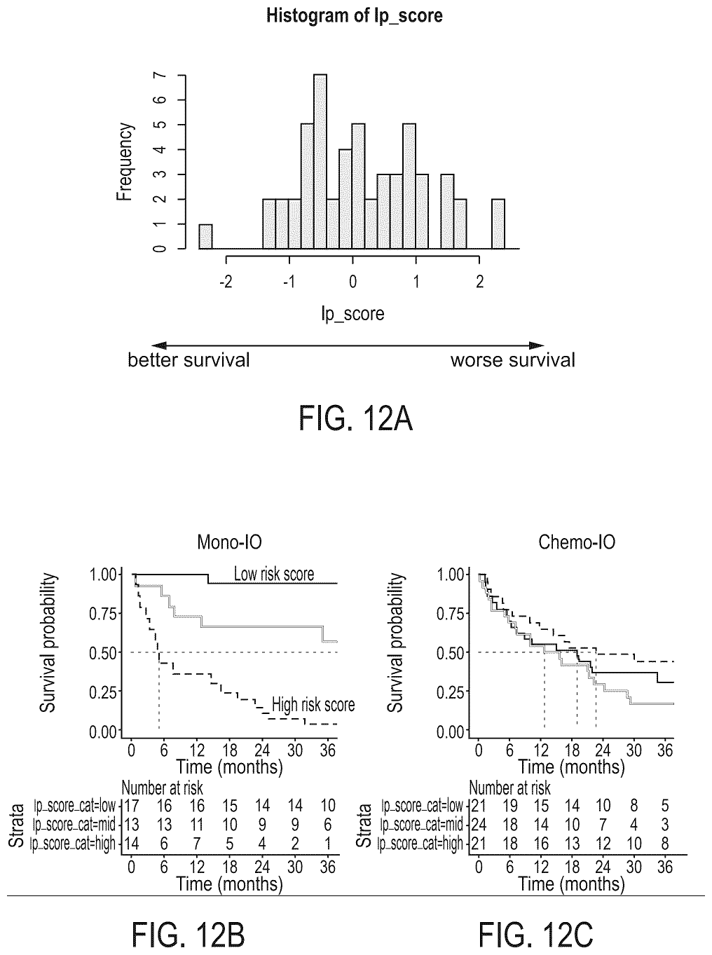

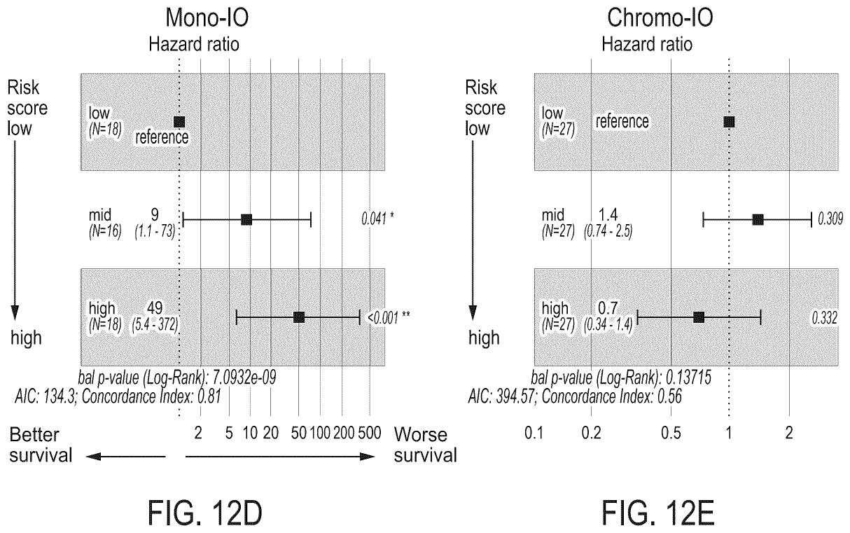

FIG. 12A illustrates a histogram 1200A of the determined for the training samples. As shown in the figure, a higher survival score was associated with a worse survival, while a lower survival score was associated with a better survival. FIG. 11B illustrates a survival plot 1200B for patients (e.g., n=125) that were treated with a mono-immunotherapy. FIG. 12C illustrates a survival plot 1200C for patients (e.g., n=263) that were treated with a mono-immunotherapy in combination with a chemo-immunotherapy. In this example, the same statistical model was applied to the data for patients treated with a mono-immunotherapy and patients treated with a mono-immunotherapy in combination with a chemo-immunotherapy. As shown in the plots, the patients were stratified to determine a survival metric using one or more thresholds based on the risk score determined by the model. In one or more examples, tertiles may be used to determine the thresholds. While the patients were stratified into three groups, a skilled artisan would understand that the patients may be stratified into more or less groups without departing from the scope of this disclosure.

A comparison of plots 1200B and 1200C yield insights into the impact of treatment on survival of a patient. For instance, patients in the first group (e.g., corresponding to a low risk score) had a better overall survival when treated with a mono-immunotherapy as opposed to treatment with chemo-immunotherapy. Based on these results, embodiments of the present disclosure may be used to predict the survival outcomes and/or a survival metric of new patient. For instance, the system may receive data associated with a new patient and determine one or more QPFs. These QPFs can be input into the statistical model to determine the risk score. Based on the risk score, the system may determine a survival metric and may further determine whether the patient is a more suitable candidate for mono-immunotherapy and/or chemotherapy. For instance, if the patient has a low risk score, the system may determine that the patient is a suitable candidate for mono-immunotherapy.

FIG. 12D illustrates the hazard ratio for the patients treated with mono-immunotherapy stratified based on the risk score. FIG. 12E illustrates the hazard ratio for the patients treated with chemo-immunotherapy stratified based on the risk score. As shown in FIG. 12D, patients treated with mono-immunotherapy who have a risk score in the second and third groups (e.g., associated with a higher risk score) are associated with a worse survival than patients in the first group. As shown in FIG. 12E, the risk score alone was not predictive of the survival outcomes for patients treated with chemo-immunotherapy.

As discussed above, FIG. 13 illustrates an exemplary box plot showing a concordance index corresponding to the three statistical models associated with the examples. As shown in the figure, a random prediction would have a concordance of about 0.5 or 50% (e.g., we would expect a random prediction to be accurate about 50% of the time). A statistical model implemented in accordance with embodiments of the present disclosure associated with FIGS. 10A-10E, e.g., using characterization features and provided with characterization features as inputs improves upon the accuracy of a random prediction. A statistical model implemented in accordance with embodiments of the present disclosure associated with FIGS. 11A-11E, e.g., that is trained using QPFs and is provided with QPFs as inputs, improves upon the accuracy of the random prediction and the genomic model. A statistical model implemented in accordance with embodiments of the present disclosure associated with FIGS. 12A-12E, e.g., that is trained using both characterization features and QPFs and is provided with both as inputs improves upon the survival predictions made by the statistical models associated with FIGS. 10A-10E (using characterization features) and the model associated with FIGS. 11A-11E (using QPFs). Accordingly, Combining the predictive power of the characterization features and the quantitative features according to embodiments of this disclosure provide a more accurate way to predict the survival of an individual compared to survival predictions based on these features alone.

### EXEMPLARY IMPLEMENTATIONS

Exemplary implementations of the methods and systems described herein include:

1. A method for predicting a survival of an individual with a disease, the method comprising:
providing a plurality of nucleic acid molecules obtained from a sample from a subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules;
receiving, at one or more processors, sequence read data for the plurality of sequence reads associated with the sample from the subject;
selecting, using the one or more processors, a plurality of sequence reads from the sequence read data;
determining, using the one or more processors, one or more characterization features based on the plurality of sequence reads;
receiving, using the one or more processors, one or more digital pathology features;
inputting, using the one or more processors, the one or more characterization features and the one or more digital pathology features into one or more statistical models; and
predicting, using the one or more processors, a survival metric indicative of a likelihood of survival of the individual, based on outputs of the one or more statistical models.

2. The method of clause 1, wherein the one or more characterization features comprise a presence of a genomic alteration, an absence of a genomic alteration, a tumor mutational burden (TMB), a PD-L1 status, a chromosome arm level, cytoband level copy number alterations, mutational signatures, copy number signatures, or a combination thereof.

3. The method of any of clause 1 or clause 2, further comprising:
receiving, using the one or more processors, one or more clinical features; and
inputting, using the one or more processors, the one or more clinical features into the one or more statistical models.

4. The method of clause 3, wherein the one or more clinical features comprises a sample type and the method further comprises pre-processing the one or more clinical features to account for bias associated with the sample type.

5. The method of clause 4, wherein the sample type comprises a tissue resection type, a core needle biopsy type, or an unknown type.

6. The method of any of clauses 1 to 5, wherein the one or more characterization features and the one or more digital pathology features are selected based on the disease.

7. The method of any of clauses 1 to 6, wherein an output of the statistical model comprises a survival score, wherein the survival score is indicative of the survival of the individual.

8. The method of clause 7, wherein the survival score is indicative of the survival of the individual when treated with a treatment.

9. The method of any of clauses 7 to 8, wherein predicting the survival metric of the individual comprises comparing the survival score to one or more predefined thresholds,
in accordance with a determination that the survival score is greater than or equal to a first predetermined threshold, determining, using the one or more processors, that the survival metric is indicative of a poor likelihood of survival; and
in accordance with a determination that the survival score is less than or equal to the first predetermined threshold, determining, using the one or more processors, that the survival metric is indicative of a strong likelihood of survival.

10. The method of any of clauses 1 to 9, wherein the subject is suspected of having or is determined to have cancer.

11. The method of clause 10, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

12. The method of clause 10, wherein the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

13. The method of clause 12, further comprising treating the subject with an anti-cancer therapy.

14. The method of clause 13, wherein the anti-cancer therapy comprises a targeted anti-cancer therapy.

15. The method of clause 14, wherein the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

16. The method of any of clauses 1 to 15, further comprising obtaining the sample from the subject.

17. The method of any one of clauses 1 to 16, wherein the sample comprises a tissue biopsy sample.

18. The method of any one of clauses 1 to 17, wherein the plurality of nucleic acid molecules comprises a mixture of tumor nucleic acid molecules and non-tumor nucleic acid molecules.

19. The method of clause 18, wherein the tumor nucleic acid molecules are derived from a tumor portion of a heterogeneous tissue biopsy sample, and the non-tumor nucleic acid molecules are derived from a normal portion of the heterogeneous tissue biopsy sample.

20. The method of any one of clauses 1 to 19, wherein the one or more adapters comprise amplification primers, flow cell adaptor sequences, substrate adapter sequences, or sample index sequences.

21. The method of any one of clauses 1 to 20, wherein the captured nucleic acid molecules are captured from the amplified nucleic acid molecules by hybridization to one or more bait molecules.

22. The method of clause 21, wherein the one or more bait molecules comprise one or more nucleic acid molecules, each comprising a region that is complementary to a region of a captured nucleic acid molecule.

23. The method of any one of clauses 1 to 22, wherein amplifying nucleic acid molecules comprises performing a polymerase chain reaction (PCR) amplification technique, a non-PCR amplification technique, or an isothermal amplification technique.

24. The method of any one of clauses 1 to 23, wherein the sequencing comprises use of a massively parallel sequencing (MPS) technique, whole genome sequencing (WGS), whole exome sequencing, targeted sequencing, direct sequencing, or Sanger sequencing technique.

25. The method of clause 24, wherein the sequencing comprises massively parallel sequencing, and the massively parallel sequencing technique comprises next generation sequencing (NGS).

26. The method of any one of clauses 1 to 25, wherein the sequencer comprises a next generation sequencer.

27. The method of any one of clauses 1 to 26, wherein one or more of the plurality of sequencing reads overlap one or more gene loci within one or more subgenomic intervals in the sample.

28. The method of clause 27, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

29. The method of clause 27 or clause 28, wherein the one or more gene loci comprise ABL1, ACVR1B, AKT1, AKT2, AKT3, ALK, ALOX12B, AMERI, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSFIR, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKB1A, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCDILG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKC1, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.

30. The method of clause 27 or clause 28, wherein the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3K8, PIGF, PTCH, RAF, RANKL, RET, ROS1, SLAMF7, VEGF, VEGFA, VEGFB, or any combination thereof.

31. The method of any one of clauses 1 to 30, further comprising generating, by the one or more processors, a report indicating the survival metric.

32. The method of clause 31, further comprising transmitting the report to a healthcare provider.

33. The method of clause 32, wherein the report is transmitted via a computer network or a peer-to-peer connection.

34. A method for predicting a survival of an individual with a disease, the method comprising:
receiving, using one or more processors, sequence read data associated with a sample from the individual;
selecting, using the one or more processors, a plurality of sequence reads from the sequence read data;
determining, using the one or more processors, one or more characterization features based on the plurality of sequence reads;
receiving, using the one or more processors, one or more digital pathology features;
inputting, using the one or more processors, the one or more characterization features and the one or more digital pathology features into one or more statistical models; and
predicting, using the one or more processors, a survival metric indicative of a likelihood of survival of the individual, based on outputs of the one or more statistical models.

35. The method of clause 34, wherein the one or more characterization features comprise a presence of a genomic alteration, an absence of a genomic alteration, a tumor mutational burden (TMB), a PD-L1 status, chromosome arm level, cytoband level copy number alterations, mutational signatures, copy number signatures, or a combination thereof.

36. The method of clause 35, wherein the PD-L1 status comprises a negative status, a positive-low status, or a positive-high status.

37. The method of clause 35, wherein the genomic alteration comprises a single nucleotide variant (SNV), insertion and/or deletion (indel), rearrangement, or copy number variant (CNV).

38. The method of any of clauses 34 to 37, wherein the one or more digital pathology features comprise a total cancer epithelium value, a regional cancer epithelium value, an area of a largest region of cancer epithelium, a perimeter of the largest region of cancer epithelium, a filled area of the largest region of cancer epithelium, a number of clusters of cells in the cancer epithelium, or a combination thereof.

39. The method of any of clauses 34 to 38, further comprising:
receiving, using the one or more processors, one or more clinical features; and
inputting, using the one or more processors, the one or more clinical features into the one or more statistical models.

40. The method of clause 39, wherein the one or more clinical features comprises a sex, a smoking status, a tissue type, a sample type, a practice type, a self-care functional performance status (ECOG performance status), an age, a stage at diagnosis, a tumor type, or a combination thereof.

41. The method of clause 40, wherein the sample type comprises a tissue resection type, a core needle biopsy type, or an unknown type.

42. The method of any of clauses 34 to 41, wherein the one or more characterization features and the one or more digital pathology features are selected based on the disease.

43. The method of any of clauses 34 to 42, wherein the one or more digital pathology features comprise quantitative pathology features (QPFs).

44. The method of any of clauses 34 to 43, wherein the one or more digital pathology features are obtained from one or more whole slide images (WSIs).

45. The method of any of clauses 39 to 44, wherein the one or more clinical features comprises the sample type and the method further comprises pre-processing the one or more clinical features to account for bias associated with the sample type.

46. The method of any of clauses 34 to 45, further comprising reducing a number of the one or more digital pathology features by associating the one more digital pathology features into one or more groups based on highly correlated features of the one or more digital pathology features.

47. The method of any one of clauses 34 to 46, wherein an output of the statistical model comprises a survival score, wherein the survival score is indicative of the survival of the individual.

48. The method of clause 47, wherein the survival score is indicative of the survival of the individual when treated with a treatment.

49. The method of any of clauses 47 to 48, wherein predicting, using the one or more processors, the survival metric of the individual comprises comparing the survival score to one or more predefined thresholds, in accordance with a determination that the survival score is greater than or equal to a first predetermined threshold, determining, using the one or more processors, that the survival metric is indicative of a poor likelihood of survival; and in accordance with a determination that the survival score is less than or equal to the first predetermined threshold, determining, using the one or more processors, that the survival metric is indicative of a strong likelihood of survival.

50. The method of clause 49, wherein the strong likelihood of survival is associated with a likelihood of survival of greater than 50% for a predetermined period of time.

51. The method of any of clauses 49 to 50, wherein the poor likelihood of survival is associated with a likelihood of survival of less than 50% for the predetermined period of time.

52. The method of any of clauses 49 to 51, further comprising determining the one or more predetermined thresholds using an F1-score, an F2-score, Mathew's correlation coefficient, Newton's index, a time-dependent ROC curve, a hazard ratio and p-value, a likelihood ratio test, or a combination thereof.

53. The method of any of clauses 34 to 52, wherein the survival metric is associated with a treatment comprising an immunotherapy.

54. The method of clause 53, wherein the immunotherapy is a monotherapy.

55. The method of clause 53, wherein the treatment comprises an immunotherapy in combination with a chemotherapy.

56. The method of clause 55, wherein the chemotherapy comprises one or more of a platinum agent and an antifolate agent.

57. The method of clause 56, wherein the platinum agent is cisplatin or carboplatin.

58. The method of any of clause 56 or clause 57, wherein the antifolate is pemetrexed.

59. The method of any of clauses 55 to 57, wherein the chemotherapy comprises cisplatin or carboplatin in combination with pemetrexed.

60. The method of any of clauses 53 to 59, wherein the treatment is a first-line treatment.

61. The method of any of clauses 53 to 60, wherein the immunotherapy comprises an immune checkpoint inhibitor (ICI).

62. The method of clause 61, wherein the ICI comprises an inhibitor of PD-1 or PD-L1.

63. The method of any of clauses 34 to 62, wherein the one or more statistical models comprises a first statistical model associated with immunotherapy as a monotherapy and a second statistical model associated with immunotherapy in combination with a chemotherapy.

64. The method of clause 63, wherein:
an output of the first statistical model corresponds to a survival score indicative of an overall survival or progression free survival of the individual when treated with the immunotherapy as monotherapy and
an output of the second statistical model corresponds to a survival score indicative of an overall survival or progression free survival of the individual when treated with the immunotherapy in combination with chemotherapy.

65. The method of any of clauses 34 to 64, wherein the individual has cancer.

66. The method of any of clauses 34 to 65, wherein the individual has non-small cell lung cancer (NSCLC).

67. The method of clause 66, wherein the individual has advanced, non-squamous NSCLC.

68. The method of clause 66 or clause 67, wherein the NSCLC does not have an alteration in an EGFR or ALK gene.

69. The method of any of clauses 34-68, further comprising training the statistical model, wherein training the statistical model comprises:
receiving, using the one or more processors, training data based on a plurality of training samples; and
training, using the one or more processors, the statistical model based on the training data to obtain a trained statistical model.

70. The method of clause 69, wherein training the statistical model comprises:
inputting, using the one or more processors, the training data into the statistical model;
determining, using the one or more processors, a score based on the training data; and
updating, using the one or more processors, one or more weights associated with the statistical model based on the score.

71. The method of clause 70, wherein the one or more weights are associated with a specific disease.

72. The method of any of clauses 69-71, further comprising filtering, using the one or more processors, the training data to select the one or more characterization features and the one or more digital pathology features for the trained statistical model based on the weights.

73. The method of any of clauses 69-72, wherein the training data comprises one or more training characterization features, one or more training digital pathology features, one or more training clinical features, one or more clinical outcomes, or a combination thereof.

74. The method of any of clauses 34 to 73, wherein the statistical model is a machine learning model.

75. The method of any of clauses 34 to 74, wherein the statistical model is part of a machine learning process.

76. The method of any of clauses 34 to 75, wherein the statistical model includes an artificial intelligence learning model.

77. The method of any of clauses 34 to 76, wherein the statistical model comprises a random forest model.

78. The method of any of clauses 34 to 77, wherein the statistical model comprises a lasso regression model.

79. The method of any of clauses 34 to 78, wherein the statistical model comprises at least one of an extreme gradient boosting model, a logistic regression model, a lasso regression model, an elastic net model, a ridge regression model, a random forest model, a support vector machine model, a k-nearest neighbor model, a Bayesian model, a naïve-based model, a Gaussian naïve-based model, a clustering model, a limited expectation maximization model, a gradient boosting ensemble model, an adaboost model, a bagging model, a neural network model, a backpropagation model, a stochastic gradient descent model, a linear regression model, a non-linear regression model, a multivariate regression model, a robust machine learning model, and a proportional hazards model.

80. The method any of clauses 34 to 79, wherein the sequence read data for the individual is based on a targeted exome sequencing panel.

81. The method any of clauses 34 to 80, wherein the sequence read data for the individual is based on a whole exome sequencing panel or a whole genome sequencing panel.

82. The method any of clauses 34 to 81, wherein the sequence read data for the individual is derived from a single biopsy sample or derived from multiple biopsy samples.

83. The method any of clauses 34 to 82, wherein the one or more characterization features comprises: a BCOR alteration, a BRAF alteration, a FAM123B alteration, a GATA3 alteration, a HGF alteration, an IRF2 alteration, a NF2 alteration, a NPM1 alteration, a PIK3CA alteration, a RAD54L alteration, a SDHC alteration, a SMAD4 alteration, and a tumor mutational burden, and wherein the one or more digital pathology features comprises a granulocyte cell count over an immune cell count, a cluster dispersion standard deviation of lymphocytes in a cancer epithelium, a cluster size standard deviation of cancer cells in the cancer epithelium, a solidity of a largest region of a cancer stroma, and a total Euler number of the cancer epithelium.

84. The method any of clauses 34 to 83, further comprising assigning, using the one or more processors, a therapy for the individual based on the predicted survival metric.

85. The method any of clauses 34 to 84 further comprising administering, using the one or more processors, a treatment to the individual based on the predicted survival metric.

86. The method any of clauses 34 to 85, further comprising associating, using the one or more processors, the individual with a clinical trial based on the predicted survival metric.

87. The method any of clauses 34 to 86, further comprising monitoring, using the one or more processors, a prognosis of the individual based on the predicted survival metric.

88. The method any of clauses 34 to 87, further comprising predicting, using the one or more processors, one or more clinical outcomes based on the predicted survival metric.

89. A method for predicting a response to treatment or a survival of an individual with a disease, the method comprising:
receiving, using one or more processors, sequence read data associated with a sample from the individual;
selecting, using the one or more processors, a plurality of sequence reads from the sequence read data;
determining, using the one or more processors, one or more characterization features based on the plurality of sequence reads;
receiving, using the one or more processors, one or more digital pathology features;
inputting, using the one or more processors, the one or more characterization features and the one or more digital pathology features into one or more statistical models; and
predicting, using the one or more processors, a survival metric indicative of a likelihood of survival of the individual or an estimated response to treatment, based on outputs of the one or more statistical models.

90. A method for predicting a response to treatment of an individual with a disease, the method comprising:
receiving, using one or more processors, sequence read data associated with a sample from the individual;
selecting, using the one or more processors, a plurality of sequence reads from the sequence read data;
determining, using the one or more processors, one or more characterization features based on the plurality of sequence reads;
receiving, using the one or more processors, one or more digital pathology features;
inputting, using the one or more processors, the one or more characterization features and the one or more digital pathology features into one or more statistical models; and
predicting, using the one or more processors, a survival metric indicative of a estimated response of the individual to the treatment, based on outputs of the one or more statistical models.

91. A method of treating a subject having a cancer comprising:
receiving, using one or more processors, sequence read data associated with a sample from the individual;
selecting, using the one or more processors, a plurality of sequence reads from the sequence read data;
determining, using the one or more processors, one or more characterization features based on the plurality of sequence reads;
receiving, using the one or more processors, one or more digital pathology features;
inputting, using the one or more processors, the one or more characterization features and the one or more digital pathology features into one or more statistical models;
predicting, using the one or more processors, a survival metric indicative of a likelihood of survival of the individual, based on outputs of the one or more statistical models;
treating the subject with a mono-immunotherapy if the survival metric is above a threshold; and
treating the subject with the mono-immunotherapy and a chemo-immunotherapy if the survival metric is below the threshold.

92. A method of selecting a treatment for a subject having a cancer comprising:
receiving, using one or more processors, sequence read data associated with a sample from the individual;
selecting, using the one or more processors, a plurality of sequence reads from the sequence read data;
determining, using the one or more processors, one or more characterization features based on the plurality of sequence reads;
receiving, using the one or more processors, one or more digital pathology features;
inputting, using the one or more processors, the one or more characterization features and the one or more digital pathology features into one or more statistical models;
predicting, using the one or more processors, a survival metric indicative of a likelihood of survival of the individual, based on outputs of the one or more statistical models;
selecting a mono-immunotherapy as the treatment if the survival metric is above a threshold; and
selecting the mono-immunotherapy and a chemo-immunotherapy as the treatment if the survival metric is below the threshold.

93. A method of identifying one or more treatment options for a subject having a cancer, the method comprising:
receiving, using one or more processors, sequence read data associated with a sample from the individual;
selecting, using the one or more processors, a plurality of sequence reads from the sequence read data;
determining, using the one or more processors, one or more characterization features based on the plurality of sequence reads;
receiving, using the one or more processors, one or more digital pathology features;
inputting, using the one or more processors, the one or more characterization features and the one or more digital pathology features into one or more statistical models;
predicting, using the one or more processors, a survival metric indicative of a likelihood of survival of the individual, based on outputs of the one or more statistical models;
identifying a mono-immunotherapy as the treatment if the survival metric is above a threshold; and
identifying the mono-immunotherapy and a chemo-immunotherapy as the treatment if the survival metric is below the threshold.

94. A method of treating a subject having a cancer comprising:
receiving, using the one or more processors, one or more digital pathology features;
inputting, using the one or more processors, the one or more digital pathology features into one or more statistical models;
predicting, using the one or more processors, a survival metric indicative of a likelihood of survival of the individual, based on outputs of the one or more statistical models;
treating the subject with a mono-immunotherapy if the survival metric is above a threshold; and
treating the subject with the mono-immunotherapy and a chemo-immunotherapy if the survival metric is below the threshold.

95. A method of selecting a treatment for a subject having a cancer comprising:
receiving, using the one or more processors, one or more digital pathology features; inputting, using the one or more processors, the one or more digital pathology features into one or more statistical models;
predicting, using the one or more processors, a survival metric indicative of a likelihood of survival of the individual, based on outputs of the one or more statistical models;
selecting a mono-immunotherapy as the treatment if the survival metric is above a threshold; and
selecting the mono-immunotherapy and a chemo-immunotherapy as the treatment if the survival metric is below the threshold.

96. A method of identifying one or more treatment options for a subject having a cancer, the method comprising:
receiving, using the one or more processors, one or more digital pathology features;
inputting, using the one or more processors, the one or more digital pathology features into one or more statistical models;
predicting, using the one or more processors, a survival metric indicative of a likelihood of survival of the individual, based on outputs of the one or more statistical models;
identifying a mono-immunotherapy as the treatment if the survival metric is above a threshold; and
identifying the mono-immunotherapy and a chemo-immunotherapy as the treatment if the survival metric is below the threshold.

97. A method of treating a subject having a cancer comprising:
receiving, using one or more processors, sequence read data associated with a sample from the individual;
selecting, using the one or more processors, a plurality of sequence reads from the sequence read data;
determining, using the one or more processors, one or more characterization features based on the plurality of sequence reads;
inputting, using the one or more processors, the one or more characterization features into one or more statistical models;
predicting, using the one or more processors, a survival metric indicative of a likelihood of survival of the individual, based on outputs of the one or more statistical models;
treating the subject with a mono-immunotherapy if the survival metric is above a threshold; and
treating the subject with the mono-immunotherapy and a chemo-immunotherapy if the survival metric is below the threshold.

98. A method of selecting a treatment for a subject having a cancer comprising:
receiving, using one or more processors, sequence read data associated with a sample from the individual;
selecting, using the one or more processors, a plurality of sequence reads from the sequence read data;
determining, using the one or more processors, one or more characterization features based on the plurality of sequence reads;
inputting, using the one or more processors, the one or more characterization features into one or more statistical models;
predicting, using the one or more processors, a survival metric indicative of a likelihood of survival of the individual, based on outputs of the one or more statistical models;
selecting a mono-immunotherapy as the treatment if the survival metric is above a threshold; and
selecting the mono-immunotherapy and a chemo-immunotherapy as the treatment if the survival metric is below the threshold.

99. A method of identifying one or more treatment options for a subject having a cancer, the method comprising:
receiving, using one or more processors, sequence read data associated with a sample from the individual;
selecting, using the one or more processors, a plurality of sequence reads from the sequence read data;
determining, using the one or more processors, one or more characterization features based on the plurality of sequence reads;
receiving, using the one or more processors, one or more digital pathology features;
inputting, using the one or more processors, the one or more characterization features and the one or more digital pathology features into one or more statistical models;
predicting, using the one or more processors, a survival metric indicative of a likelihood of survival of the individual, based on outputs of the one or more statistical models;
identifying a mono-immunotherapy as the treatment if the survival metric is above a threshold; and
identifying the mono-immunotherapy and a chemo-immunotherapy as the treatment if the survival metric is below the threshold.

100. The method of any one of clauses 91-99, wherein the chemotherapy comprises one or more of a platinum agent and an antifolate agent.

101. The method of clause 100, wherein the platinum agent is cisplatin or carboplatin.

102. The method of clause 100 or clause 101, wherein the antifolate is pemetrexed.

103. The method of any one of clauses 100-102, wherein the chemotherapy comprises cisplatin or carboplatin in combination with pemetrexed.

104. The method of any one of clauses 100-103, wherein the mono-immunotherapy comprises an immune checkpoint inhibitor (ICI).

105. The method of clause 104, wherein the ICI comprises an inhibitor of PD-1 or PD-L1.

106. The method of any one of clauses 91-105, wherein the cancer comprises lung cancer.

107. The method of clause 106, wherein the individual has non-squamous non-small cell lung cancer (NSCLC) or advanced, non-squamous NSCLC.

108. The method of any one of clauses 91-107, wherein the one or more characterization features comprises a BCOR alteration, a BRAF alteration, a FAM123B alteration, a GATA3 alteration, a HGF alteration, an IRF2 alteration, a NF2 alteration, a NPM1 alteration, a PIK3CA alteration, a RAD54L alteration, a SDHC alteration, a SMAD4 alteration, and a tumor mutational burden.

109. The method of any one of clauses 91-108, wherein the one or more digital pathology features comprises a granulocyte cell count over an immune cell count, a cluster dispersion standard deviation of lymphocytes in a cancer epithelium; a cluster size standard deviation of cancer cells in the cancer epithelium, a solidity of a largest region of a cancer stroma, and a total Euler number of the cancer epithelium.

110. A method for diagnosing a disease, the method comprising:
diagnosing that a subject has the disease based on a determination of a survival metric indicative of a likelihood of survival of the subject for a sample from the subject, wherein survival metric is determined according to the method of any one of clauses 1 to 105.

111. A method of selecting an anti-cancer therapy, the method comprising:
responsive to determining a survival metric indicative of a likelihood of survival of the subject for a sample from a subject, selecting an anti-cancer therapy for the subject, wherein survival metric is determined according to the method of any one of clauses 1 to 105.

112. A method of treating a cancer in a subject, comprising:
responsive to determining survival metric indicative of a likelihood of survival of the subject for a sample from the subject, administering an effective amount of an anti-cancer therapy to the subject, wherein survival metric is determined according to the method of any one of clauses 1 to 105.

113. A method for monitoring cancer progression or recurrence in a subject, the method comprising:
determining a first survival metric indicative of a likelihood of survival of the subject in a first sample obtained from the subject at a first time point according to the method of any one of clauses 1 to 105;
determining a second survival metric in a second sample obtained from the subject at a second time point; and comparing the first survival metric to the second survival metric, thereby monitoring the cancer progression or recurrence.

114. The method of clause 113, wherein the second survival metric for the second sample is determined according to the method of any one of clauses 1 to 109.

115. The method of clause 113 or clause 114, further comprising selecting an anti-cancer therapy for the subject in response to the cancer progression.

116. The method of clause 113 or clause 114, further comprising administering an anti-cancer therapy to the subject in response to the cancer progression.

117. The method of clause 113 or clause 114, further comprising adjusting an anti-cancer therapy for the subject in response to the cancer progression.

118. The method of any one of clauses 115 to 117, further comprising adjusting a dosage of the anti-cancer therapy or selecting a different anti-cancer therapy in response to the cancer progression.

119. The method of clause 118, further comprising administering the adjusted anti-cancer therapy to the subject.

120. The method of any one of clauses 113 to 119, wherein the first time point is before the subject has been administered an anti-cancer therapy, and wherein the second time point is after the subject has been administered the anti-cancer therapy.

121. The method of any one of clauses 113 to 120, wherein the subject has a cancer, is at risk of having a cancer, is being routine tested for cancer, or is suspected of having a cancer.

122. The method of any one of clauses 113 to 121, wherein the cancer is a solid tumor.

123. The method of any one of clauses 113 to 121, wherein the cancer is a hematological cancer.

124. The method of any one of clauses 113 to 123, wherein the anti-cancer therapy comprises chemotherapy, radiation therapy, immunotherapy, a targeted therapy, or surgery.

125. The method of any one of clauses 1 to 109, further comprising determining, identifying, or applying the value of a survival metric indicative of a likelihood of survival of the subject for the sample as a diagnostic value associated with the sample.

126. The method of any one of clauses 1 to 109, further comprising generating a genomic profile for the subject based on the determination of a survival metric indicative of a likelihood of survival of the subject.

127. The method of clause 126, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.

128. The method of clause 126 or clause 127, wherein the genomic profile for the subject further comprises results from a nucleic acid sequencing-based test.

129. The method of any one of clauses 126 to 128, further comprising selecting an anti-cancer therapy, administering an anti-cancer therapy, or applying an anti-cancer therapy to the subject based on the generated genomic profile.

130. The method of any one of clauses 1 to 109, wherein the determination of a survival metric indicative of a likelihood of survival of the subject for the sample is used in making suggested treatment decisions for the subject.

131. The method of any one of clauses 1 to 109, wherein the determination of a survival metric indicative of a likelihood of survival of the subject or the sample is used in applying or administering a treatment to the subject.

132. A system comprising:
one or more processors; and
a memory communicatively coupled to the one or more processors and configured to store instructions that, when executed by the one or more processors, cause the system to perform a method comprising:
   receiving, using the one or more processors, sequence read data associated with a sample from the individual;
   selecting, using the one or more processors, a plurality of sequence reads from the sequence read data;
   determining, using the one or more processors, one or more characterization features based on the plurality of sequence reads;
   receiving, using the one or more processors, one or more digital pathology features;
   inputting, using the one or more processors, the one or more characterization features and the one or more digital pathology features into one or more statistical models; and
   predicting, using the one or more processors, a survival metric indicative of a likelihood of survival of the individual, based on outputs of the one or more statistical models.

133. A non-transitory computer-readable storage medium storing one or more programs, the one or more programs comprising instructions, which when executed by one or more processors of a system, cause the system to perform a method comprising:
receiving, using the one or more processors, sequence read data associated with a sample from the individual;
selecting, using the one or more processors, a plurality of sequence reads from the sequence read data;
determining, using the one or more processors, one or more characterization features based on the plurality of sequence reads;
receiving, using the one or more processors, one or more digital pathology features;
inputting, using the one or more processors, the one or more characterization features and the one or more digital pathology features into one or more statistical models; and
predicting, using the one or more processors, a survival metric indicative of a likelihood of survival of the individual, based on outputs of the one or more statistical models.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

What is claimed is:

1. A method for predicting a survival of an individual with a disease, the method comprising:
receiving, using one or more processors, sequence read data associated with a sample from the individual;
selecting, using the one or more processors, a plurality of sequence reads from the sequence read data;
determining, using the one or more processors, one or more characterization features based on the plurality of sequence reads;
receiving, using the one or more processors, one or more digital pathology features;
inputting, using the one or more processors, the one or more characterization features and the one or more digital pathology features into one or more statistical models; and
predicting, using the one or more processors, a survival metric indicative of a likelihood of survival of the individual, based on outputs of the one or more statistical models.

2. The method of claim 1, wherein the one or more characterization features comprise a presence of a genomic alteration, an absence of a genomic alteration, a tumor mutational burden (TMB), a PD-L1 status, chromosome arm level, cytoband level copy number alterations, mutational signatures, copy number signatures, or a combination thereof.

3. The method of claim 2, wherein the PD-L1 status comprises a negative status, a positive-low status, or a positive-high status, and/or wherein the genomic alteration comprises a single nucleotide variant (SNV), insertion and/or deletion (indel), rearrangement, or copy number variant (CNV).

4. The method of claim 1, wherein the one or more digital pathology features comprise a total cancer epithelium value, a regional cancer epithelium value, an area of a largest region of cancer epithelium, a perimeter of the largest region of cancer epithelium, a filled area of the largest region of cancer epithelium, a number of clusters of cells in the cancer epithelium, or a combination thereof.

5. The method of claim 1, further comprising:
receiving, using the one or more processors, one or more clinical features; and
inputting, using the one or more processors, the one or more clinical features into the one or more statistical models,
wherein the one or more clinical features comprises a sex, a smoking status, a tissue type, a sample type, a practice type, a self-care functional performance status (ECOG performance status), an age, a stage at diagnosis, a tumor type, or a combination thereof.

6. The method of claim 1, wherein the one or more characterization features and the one or more digital pathology features are selected based on the disease.

7. The method of claim 1, wherein the one or more digital pathology features comprise quantitative pathology features (QPFs).

8. The method of claim 1, wherein the one or more digital pathology features are obtained from one or more whole slide images (WSIs).

9. The method of claim 1, further comprising reducing a number of the one or more digital pathology features by associating the one more digital pathology features into one or more groups based on highly correlated features of the one or more digital pathology features.

10. The method of claim 1, wherein an output of the statistical model comprises a survival score, wherein the survival score is indicative of the survival of the individual.

11. The method of claim 10, wherein predicting, using the one or more processors, the survival metric of the individual comprises comparing the survival score to one or more predefined thresholds,
in accordance with a determination that the survival score is greater than or equal to a first predetermined threshold, determining, using the one or more processors, that the survival metric is indicative of a likelihood of survival of less than 50% for a predetermined period of time; and
in accordance with a determination that the survival score is less than or equal to the first predetermined threshold, determining, using the one or more processors, that the survival metric is indicative of a likelihood of survival of greater than 50% for the predetermined period of time.

12. The method of claim 1, wherein the survival metric is associated with a treatment comprising an immunotherapy.

13. The method of claim 1, wherein the one or more statistical models comprises a first statistical model associated with immunotherapy as a monotherapy and a second statistical model associated with immunotherapy in combination with a chemotherapy.

14. The method of claim 13, wherein:
an output of the first statistical model corresponds to a survival score indicative of an overall survival or progression free survival of the individual when treated with the immunotherapy as monotherapy and
an output of the second statistical model corresponds to a survival score indicative of an overall survival or progression free survival of the individual when treated with the immunotherapy in combination with chemotherapy.

15. The method of claim 1, further comprising training the statistical model, wherein training the statistical model comprises:
receiving, using the one or more processors, training data based on a plurality of training samples; and
training, using the one or more processors, the statistical model based on the training data to obtain a trained statistical model.

16. The method of claim 15, wherein training the statistical model comprises:
inputting, using the one or more processors, the training data into the statistical model;
determining, using the one or more processors, a score based on the training data; and
updating, using the one or more processors, one or more weights associated with the statistical model based on the score, wherein the one or more weights are associated with a specific disease.

17. The method of claim 15, further comprising filtering, using the one or more processors, the training data to select the one or more characterization features and the one or more digital pathology features for the trained statistical model based on the weights.

18. The method of claim 15, wherein the training data comprises one or more training characterization features, one or more training digital pathology features, one or more training clinical features, one or more clinical outcomes, or a combination thereof.

19. A method for predicting a response to treatment or a survival of an individual with a disease, the method comprising:
receiving, using one or more processors, sequence read data associated with a sample from the individual;
selecting, using the one or more processors, a plurality of sequence reads from the sequence read data;
determining, using the one or more processors, one or more characterization features based on the plurality of sequence reads;
receiving, using the one or more processors, one or more digital pathology features;
inputting, using the one or more processors, the one or more characterization features and the one or more digital pathology features into one or more statistical models; and
predicting, using the one or more processors, based on outputs of the one or more statistical models, a survival metric indicative of one or more of:
a likelihood of survival of the individual or an estimated response to treatment, and
an estimated response of the individual to the treatment.

20. A method of treating an individual with a disease, the method comprising:
receiving, using one or more processors, sequence read data associated with a sample from the individual;
selecting, using the one or more processors, a plurality of sequence reads from the sequence read data;
determining, using the one or more processors, one or more characterization features based on the plurality of sequence reads;
receiving, using the one or more processors, one or more digital pathology features;
inputting, using the one or more processors, the one or more characterization features and the one or more digital pathology features into one or more statistical models;
predicting, using the one or more processors, a survival metric indicative of a likelihood of survival of the individual, based on outputs of the one or more statistical models;
if the survival metric is above a threshold:
identifying a mono-immunotherapy as the treatment, and
treating the subject with the mono-immunotherapy; and
if the survival metric is below the threshold:
identifying the mono-immunotherapy and a chemo-immunotherapy as the treatment, and
treating the subject with the mono-immunotherapy and the chemo-immunotherapy.
